(19) United States
(12) Patent Application Publication
(10) Pub. No.: US 2004/0176425 A1
(43) Pub. Date: Sep. 9, 2004

(54) Cycloalkyl containing anilide ligands for the thyroid receptor

(76) Inventors: William N. Washburn, Titusville, NJ (US); Wei Meng, Pennington, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/764,118

(22) Filed: Jan. 23, 2004

Related U.S. Application Data
(60) Provisional application No. 60/442,659, filed on Jan. 24, 2003.

(51) Int. Cl. A61K 31/44; A61K 31/41; A61K 31/195; A61K 31/277

(52) U.S. Cl. 514/357; 514/381; 514/562; 514/563; 514/522; 546/330; 546/331; 562/430; 562/450

(57) ABSTRACT

Novel thyroid receptor ligands are provided having the general formula I

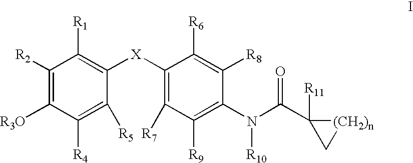

wherein

X, R₁, R₂, R₃, R₄, R₅, R₆, R₇, R₈, R₉, R₁₀ and R₁₁ are as defined herein.

In addition, a method is provided for preventing, inhibiting or treating diseases or disorders associated with metabolic dysfunction or which are dependent upon the expression of a T₃ regulated gene, wherein a compound as described above is administered in a therapeutically effective amount.

### CROSS-REFERENCE TO RELATED APPLICATION

This application claims the benefit of U.S. Provisional Application No. 60/442,659, filed Jan. 24, 2003, which is incorporated herein by reference.

### FIELD OF THE INVENTION

This invention relates to novel compounds which are thyroid receptor ligands, and to methods of preparing such compounds and to methods for using such compounds such as in the regulation of metabolism.

### BACKGROUND OF THE INVENTION

While the extensive role of thyroid hormones in regulating metabolism in humans is well recognized, the discovery and development of new specific drugs for improving the treatment of hyperthyroidism and hypothyroidism has been slow. This has also limited the development of thyroid agonists and antagonists for treatment of other important clinical indications, such as hypercholesterolemia, obesity and cardiac arrhythmias.

Thyroid hormones affect the metabolism of virtually every cell of the body. At normal levels, these hormones maintain body weight, metabolic rate, body temperature and mood, and influence blood levels of serum low density lipoprotein (LDL). Thus, in hypothyroidism there is weight gain, high levels of LDL cholesterol, and depression. In hyperthyroidism, these hormones lead to weight loss, hypermetabolism, lowering of serum LDL levels, cardiac arrhythmias, heart failure, muscle weakness, bone loss in postmenopausal women, and anxiety.

Thyroid hormones are currently used primarily as replacement therapy for patients with hypothyroidism. Therapy with L-thyroxine returns metabolic functions to normal and can easily be monitored with routine serum measurements of levels of thyroid-stimulating hormone (TSH), thyroxine (3,5,3′,5′-tetraiodo-L-thyronine, or T₄) and triiodothyronine (3,5,3′-triiodo-L-thyronine, or T₃). However, replacement therapy, particularly in older individuals, may be restricted by certain detrimental effects from thyroid hormones.

In addition, some effects of thyroid hormones may be therapeutically useful in non-thyroid disorders if adverse effects can be minimized or eliminated. These potentially useful influences include weight reduction, lowering of serum LDL levels, amelioration of depression and stimulation of bone formation. Prior attempts to utilize thyroid hormones pharmacologically to treat these disorders have been limited by manifestations of hyperthyroidism, and in particular by cardiovascular toxicity.

Furthermore, useful thyroid agonist drugs should minimize the potential for undesired consequences due to locally induced hypothyroidism, i.e. sub-normal levels of thyroid hormone activity in certain tissues or organs. This can arise because increased circulating thyroid hormone agonist concentrations may cause the pituitary to suppress the secretion of thyroid stimulating hormone (TSH), thereby reducing thyroid hormone synthesis by the thyroid gland (negative feedback control). Since endogenous thyroid hormone levels are reduced, localized hypothyroidism can result wherever the administered thyroid agonist drug fails to compensate for the reduction in endogenous hormone levels in specific tissues. For example, if the thyroid agonist drug does not penetrate the blood-brain barrier, the effects of TSH suppression can lead to CNS hypothyroidism and associated risks such as depression.

Development of specific and selective thyroid hormone receptor ligands, particularly agonists of the thyroid hormone receptor could lead to specific therapies for these common disorders, while avoiding the cardiovascular and other toxicity of native thyroid hormones. Tissue-selective thyroid hormone agonists may be obtained by selective tissue uptake or extrusion, topical or local delivery, targeting to cells through other ligands attached to the agonist and targeting receptor subtypes. Tissue selectivity can also be achieved by selective regulation of thyroid hormone responsive genes in a tissue specific manner.

Accordingly, the discovery of compounds that are thyroid hormone receptor ligands, particularly selective agonists of the thyroid hormone receptor, may demonstrate a utility for the treatment or prevention of diseases or disorders associated with thyroid hormone activity, for example: (1) replacement therapy in elderly subjects with hypothyroidism who are at risk for cardiovascular complications; (2) replacement therapy in elderly subjects with subclinical hypothyroidism who are at risk for cardiovascular complications; (3) obesity; (4) hypercholesterolemia due to elevations of plasma LDL levels; (5) depression; and (6) osteoporosis in combination with a bone resorption inhibitor.

### SUMMARY OF THE INVENTION

In accordance with the present invention, compounds are provided which are thyroid hormone receptor ligands, and have the general formula I:

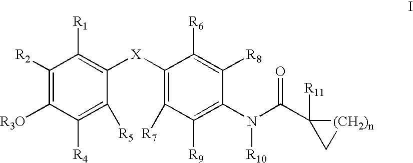

wherein

X is selected from oxygen (—O—), selenium (—Se), sulfur (—S—), sulfenyl (SO), sulfonyl (SO₂), carbonyl (—CO—), methylene (—CH₂—) and —NH—;

R₁ is selected from hydrogen, halogen, CF₃ and C₁ to C₆ alkyl;

R₂ is selected from halogen, CF₃, C₁ to C₆ alkyl, C₂ to C₆ alkenyl, C₂ to C₆ alkynyl, C₃ to C₇ cycloalkyl, C₄ to C₇ cycloalkenyl, aryl, heteroaryl, alkoxy, aryloxy, heteroaryloxy, arylalkoxy, cycloalkoxy, N(R₁₂)COR₁₃, CO(NR₁₂R₁₃), N(R₁₂) SO₂R₁₃, SO₂ (NR₁₂R₁₃), SR₁₄, SOR₁₄, SO₂R₁₄, COR₁₄, CR₁₂(OR₅)R₁₃ and CH₂NR₁₂R₁₃;

R₃ is selected from hydrogen, alkyl, benzyl, aroyl and alkanoyl;

R₄ and R₅ are each independently selected from hydrogen, halogen and alkyl;

R₆ and R₇ are each independently selected from hydrogen, halogen, cyano, C₁ to C₄ alkyl and C₃ to C₆ cycloalkyl, at least one of R₆ and R₇ being other than hydrogen;

R₈ and R₉ are each independently selected from hydrogen, halogen, alkoxy, hydroxy, cyano, CF₃ and alkyl;

R₁₀ is hydrogen or alkyl;

R₁₁ is CO₂R₁₃ or tetrazole;

R₁₂ and R₁₃ for each occurrence are each independently selected from hydrogen, alkyl, cycloalkyl, aryl, heteroaryl, arylalkyl and heteroarylalkyl;

R₁₄ is selected from alkyl, cycloalkyl, aryl, heteroaryl, arylalkyl and heteroarylalkyl; and

n is an integer from 1 to 4.

The definition of formula I above includes all prodrug-esters, stereoisomers and pharmaceutically. acceptable salts of formula I.

The compounds of formula I are thyroid hormone receptor ligands and include compounds which are, for example, selective agonists, partial agonists, antagonists or partial antagonists of the thyroid receptor. Preferably, the compounds of formula I possess activity as agonists of the thyroid receptor and may be used in the treatment of diseases or disorders associated with thyroid receptor activity. In particular, the compounds of formula I may be used in the treatment of diseases or disorders associated with metabolic dysfunction or which are dependent upon the expression of a T₃ regulated gene, such as obesity, hypercholesterolemia, atherosclerosis, cardiac arrhythmias, depression, osteoporosis, hypothyroidism, goiter, thyroid cancer, glaucoma, skin disorders or diseases and congestive heart failure.

The present invention provides for compounds of formula I, pharmaceutical compositions employing such compounds and for methods of using such compounds. In particular, the present invention provides for a pharmaceutical composition comprising a therapeutically effective amount of a compound of formula I, alone or in combination with a pharmaceutically acceptable carrier.

Further, in accordance with the present invention, a method is provided for preventing, inhibiting or treating the progression or onset of diseases or disorders associated with the thyroid receptor, such as the diseases or disorders defined above and hereinafter, wherein a therapeutically effective amount of a compound of formula I is administered to a mammalian, i.e., human patient in need of treatment.

The compounds of the invention can be used alone, in combination with other compounds of the present invention, or in combination with one or more other agent(s) active in the therapeutic areas described herein.

In addition, a method is provided for preventing, inhibiting or treating the diseases as defined above and hereinafter, wherein a therapeutically effective amount of a combination of a compound of formula I and another compound of the invention and/or another type of therapeutic agent, is administered to a mammalian patient in need of treatment.

### DETAILED DESCRIPTION OF THE INVENTION

[1] Thus, in a first embodiment, the present invention provides for a compound of formula I

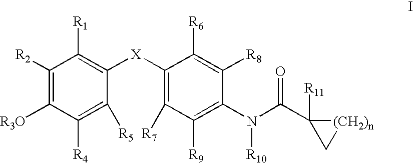

wherein:

X is selected from oxygen (—O—), selenium (—Se—), sulfur (—S—), sulfenyl (SO), sulfonyl (SO₂), carbonyl (—CO), methylene (—CH₂—) and —NH—;

R₁ is selected from hydrogen, halogen, CF₃ and C₁ to C₆ alkyl;

R₂ is selected from halogen, CF₃, C₁ to C₆ alkyl, C₂ to C₆ alkenyl, C₂ to C₆ alkynyl, C₃ to C₇ cycloalkyl, C₄ to C₇ cycloalkenyl, aryl, heteroaryl, alkoxy, aryloxy, heteroaryloxy, arylalkoxy, cycloalkoxy, N(R₁₂)COR₁₃, CO(NR₁₂R₁₃), N(R₁₂)SO₂R₁₃, SO₂(NR₁₂R₁₃), SR₁₄, SOR₁₄, SO_{2R}₁₄, COR₁₄, CR₁₂(OR₅)R₁₃ and CH₂NR₁₂R₁₃;

R₃ is selected from hydrogen, alkyl, benzyl, aroyl and alkanoyl;

R₄ and R₅ are each independently selected from hydrogen, halogen and alkyl;

R₆ and R₇ are each independently selected hydrogen, halogen, cyano, C₁ to C₄ alkyl and C₃ to C₆ cycloalkyl, where at least one of R₆ and R₇ is other than hydrogen;

R₈ and R₉ are each independently selected from hydrogen, halogen, alkoxy, hydroxy, cyano, CF₃ and alkyl;

R₁₀ is hydrogen or alkyl;

R₁₁ is CO₂R₁₃ or tetrazole;

R₁₂ and R₁₃ for each occurrence are each independently selected from hydrogen, alkyl, cycloalkyl, aryl, heteroaryl, arylalkyl and heteroarylalkyl;

R₁₄ is selected from alkyl, cycloalkyl, aryl, heteroaryl, arylalkyl and heteroarylalkyl; and

n is an integer from 1 to 4,

including all prodrugs, stereoisomers and pharmaceutically acceptable salts thereof.

[2] In a preferred embodiment, the present invention provides a compound of formula I, including all prodrugs, stereoisomers and pharmaceutically acceptable salts wherein: X is oxygen.

[3] In another preferred embodiment, the present invention provides a compound of formula I, including all prodrugs, stereoisomers and pharmaceutically acceptable salts wherein:

R₁ is hydrogen;

R₂ is C₁ to C₆ alkyl or C₃ to C₇ cycloalkyl;

R₃ is hydrogen;

R₄ is hydrogen, halogen or alkyl;

R₅ is hydrogen;

R₆ and R₇ are each independently bromo, chloro or C₁ to C₄ alkyl;

R₈ is hydrogen, halogen or alkyl;

R₉ is hydrogen or halogen;

R₁₀ is hydrogen;

R₁₁ is carboxyl; and

n is 2 or 3.

[4] In another preferred embodiment, the present invention provides a compound of formula I, including all prodrugs, stereoisomers and pharmaceutically acceptable salts wherein:

R₂ is isopropyl.

[5] In another preferred embodiment, the present invention provides a compound of formula I, including all prodrugs, stereoisomers and pharmaceutically acceptable salts wherein:

R₁ is hydrogen;

R₂ is isopropyl;

R₃ is hydrogen;

R₄ is chloro or C₁ to C₄ alkyl;

R₅ is hydrogen;

R₆ and R₇ are each independently bromo, chloro or methyl;

R₈ is hydrogen, chloro or C₁ to C₄ alkyl;

R₉ is hydrogen;

R₁₀ is hydrogen;

R₁₁ is carboxyl; and

n is 2.

[6] In another preferred embodiment, the present invention provides a compound of formula I, including all prodrugs, stereoisomers and pharmaceutically acceptable salts wherein:

R₁ is hydrogen;

R₂ is isopropyl;

R₃ is hydrogen;

R₄ is chloro or methyl;

R₅ is hydrogen;

R₆ and R₇ are bromo;

R₈ is hydrogen or methyl;

R₉ is hydrogen;

R₁₀ is hydrogen;

R₁₁ is carboxyl; and

n is 2.

[7] In a more preferred embodiment, the present invention provides a compound of formula I, including all prodrugs, stereoisomers and pharmaceutically acceptable salts selected from:

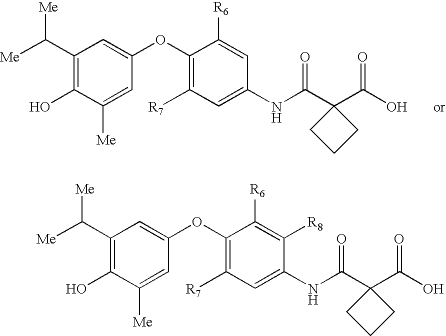

or an alkyl ester thereof.

[8] In another more preferred embodiment, the present invention provides a compound of formula I, including all prodrugs, stereoisomers and pharmaceutically acceptable salts selected from:

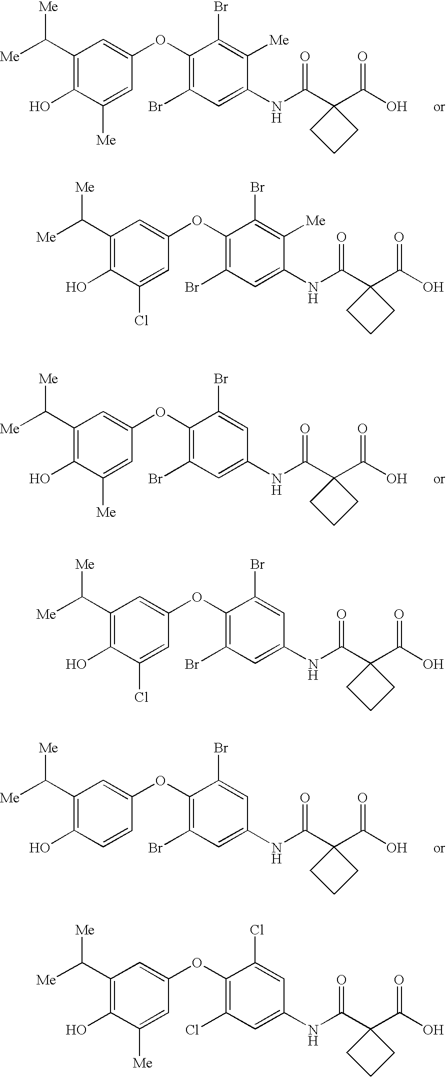

or

or an alkyl ester thereof.

[9] In another more preferred embodiment, the present invention provides a compound of formula I, including all prodrugs, stereoisomers and pharmaceutically acceptable salts selected from:

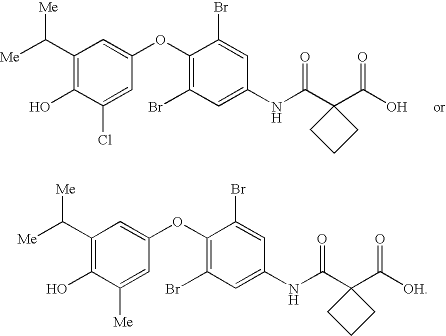

[10] In a second embodiment, the present invention provides a pharmaceutical composition comprising a compound of formula I as defined above and a pharmaceutically acceptable carrier therefor.

[11] In a preferred embodiment, the present invention provides a pharmaceutical composition as defined above further comprising at least one additional therapeutic agent selected from other compounds of formula I, anti-diabetic agents, anti-osteoporosis agents, anti-obesity agents, growth promoting agents, anti-inflammatory agents, anti-anxiety agents, anti-depressants, anti-hypertensive agents, cardiac glycosides, cholesterol/lipid lowering agents, appetite supressants, bone resorption inhibitors, thyroid mimetics, anabolic agents, anti-tumor agents and retinoids.

[12] In another preferred embodiment, the present invention provides a pharmaceutical composition as defined above wherein said additional therapeutic agent is an antidiabetic agent selected from a biguanide, a glucosidase inhibitor, a meglitinide, a sulfonylurea, a thiazolidinedione, a PPAR-alpha agonist, a PPAR-gamma agonist, a PPAR alpha/gamma dual agonist, an SGLT2 inhibitor, a glycogen phosphorylase inhibitor, an aP2 inhibitor, a glucagon-like peptide-1 (GLP-1), a dipeptidyl peptidase IV inhibitor and insulin.

[13] In another preferred embodiment, the present invention provides a pharmaceutical composition as defined above wherein said additional therapeutic agent is an antidiabetic agent selected from metformin, glyburide, glimepiride, glipyride, glipizide, chlorpropamide, gliclazide, acarbose, miglitol, troglitazone, pioglitazone, englitazone, darglitazone, rosiglitazone and insulin.

[14] In another preferred embodiment, the present invention provides a pharmaceutical composition as defined above wherein said additional therapeutic agent is an anti-obesity agent selected from an aP2 inhibitor, a PPAR gamma antagonist, a PPAR delta agonist, a beta 3 adrenergic agonist, a lipase inhibitor, a serotonin reuptake inhibitor, a cannabinoid-1 receptor antagonist and an anorectic agent.

[15] In another preferred embodiment, the present invention provides a pharmaceutical composition as defined above wherein said additional therapeutic agent is a hypolipidemic agent selected from a thiazolidinedione, an MTP inhibitor, a squalene synthetase inhibitor, an HMG CoA reductase inhibitor, a fibric acid derivative, an ACAT inhibitor, a cholesterol absorption inhibitor, an ileal Na⁺/bile cotransporter inhibitor, a bile acid sequestrant and a nicotinic acid or a derivative thereof.

[16] In a third embodiment, the present invention provides a method for preventing, inhibiting or treating a disease associated with metabolic dysfunction, or which is dependent on the expression of a T₃ regulated gene, which comprises administering to a mammalian patient in need of treatment a therapeutically effective amount of a compound of formula I.

[17] In a preferred embodiment, the present invention provides a method as defined above method for treating or delaying the progression or onset of obesity, hypercholesterolemia, atherosclerosis, depression, osteoporosis, hypothyroidism, subclinical hyperthyroidism, non-toxic goiter, reduced bone mass, density or growth, eating disorders, reduced cognitive function, thyroid cancer, glaucoma, cardiac arrhythmia, congestive heart failure or a skin disorder or disease, which comprises administering to mammalian patient in need of treatment a therapeutically effective amount of a compound of formula I.

[18] In another preferred embodiment, the present invention provides a method as defined above wherein the skin disorder or disease is dermal atrophy, post surgical bruising caused by laser resurfacing, keloids, stria, cellulite, roughened skin, actinic skin damage, lichen planus, ichtyosis, acne, psoriasis, Dernier's disease, eczema, atopic dermatitis, chloracne, pityriasis or skin scarring.

[19] In another preferred embodiment, the present invention provides a method as defined above further comprising administering, concurrently or sequentially, a therapeutically effective amount of at least one additional therapeutic agent selected from other compounds of formula I, anti-diabetic agents, anti-osteoporosis agents, anti-obesity agents, growth promoting agents, anti-inflammatory agents, anti-anxiety agents, anti-depressants, anti-hypertensive agents, cardiac glycosides, cholesterol/lipid lowering agents, appetite supressants, bone resorption inhibitors, thyroid mimetics, anabolic agents, anti-tumor agents and retinoids.

[20] In another preferred embodiment, the present invention provides a method of treating or delaying the progression or onset of a skin disorder or disease which comprises administering to a mammalian patient a therapeutically effective amount of a compound of formula I in combination with a retinoid or a vitamin D analog.

[22] In another preferred embodiment, the present invention provides a method for treating or delaying the progression or onset of obesity which comprises administering to mammalian patient in need of treatment a therapeutically effective amount of a compound of formula I.

[22] In another preferred embodiment, the present invention provides a method as defined above further comprising administering, concurrently or sequentially, a therapeutically effective amount of at least one additional therapeutic agent selected from an anti-obesity agent and an appetite suppressant.

[23] In another preferred embodiment, the present invention provides a method as defined above wherein said anti-obesity agent is selected from aP2 inhibitors, PPAR gamma antagonists, PPAR delta agonists, beta 3 adrenergic agonists, lipase inhibitors, serotonin (and dopamine) reuptake inhibitors, cannabinoid-l receptor antagonists, other thyroid receptor agents and anorectic agents.

[24] In a fourth embodiment, the present invention provides a pharmaceutical composition which functions as a selective agonist of the thyroid hormone receptor comprising a compound of formula I.

The following abbreviations are employed herein:

| | |
|---|---|
| Ph = phenyl | K₂CO₃ = potassium carbonate |
| Bn = benzyl | NaHCO₃ = sodium bicarbonate |
| t-Bu = tertiary butyl | Ph₃P = triphenylphosphine |
| Me = methyl | Ar = argon |
| Et = ethyl | N₂ = nitrogen |
| THF = tetrahydrofuran | min = minute(s) |
| Et₂O = diethyl ether | h or hr = hour(s) |
| EtOAc = ethyl acetate | L = liter |
| DMF = dimethyl formamide | mL = milliliter |
| MeOH = methanol | μL = microliter |
| EtOH = ethanol | g = gram(s) |
| i-PrOH = isopropanol | mg = milligram(s) |
| HOAc or AcOH = acetic acid | mol = moles |
| TFA = trifluoroacetic acid | mmol = millimole(s) |
| i-Pr₂NEt = diisopropylethylamine | meq = milliequivalent |
| Et₃N = triethylamine | RT = room temperature |
| DMAP = 4-dimethylaminopyridine | sat or sat'd = saturated |
| NaBH₄ = sodium borohydride | aq. = aqueous |
| KOH = potassium hydroxide | NMR = nuclear magnetic |
| NaOH = sodium hydroxide | resonance |
| LiOH = lithium hydroxide | |

EDC (or EDC.HCl) or EDCI (or EDCI.HCl) or EDAC=3-ethyl-3′-(dimethylamino)propyl-carbodiimide hydrochloride (or 1-(3-dimethylaminopropyl)-3-ethylcarbodiimide hydrochloride) HOBT or HOBT.H₂O=1-hydroxybenzotriazole hydrate HOAT=1-Hydroxy-7-azabenzotriazole TLC=thin layer chromatography HPLC=high performance liquid chromatography LC/MS=high performance liquid chromatography/mass spectrometry MS or Mass Spec=mass spectrometry

The following definitions apply to the terms as used throughout this specification, unless otherwise limited in specific instances.

The term “thyroid receptor ligand” as used herein is intended to cover any moiety which binds to a thyroid receptor. The ligand may act as an agonist, an antagonist, a partial agonist or a partial antagonist. Another term for “thyroid receptor ligand” is “thyromimetic”.

Unless otherwise indicated, the term “alkyl” as employed herein alone or as part of another group includes both straight and branched chain hydrocarbons, containing 1 to 12 carbons (in the case of alkyl or alk), in the normal chain, preferably 1 to 4 carbons, such as methyl, ethyl, propyl, isopropyl, butyl, t-butyl, or isobutyl, pentyl, hexyl, isohexyl, heptyl, 4,4-dimethylpentyl, octyl, 2,2,4-trimethylpentyl, nonyl, decyl, undecyl, dodecyl. As defined and claimed herein, the term “alkyl” includes alkyl groups as defined above optionally substituted with 1 to 4 substituents which may halo, for example F, Br, Cl or I or CF₃, alkyl, alkoxy, aryl, aryloxy, aryl(aryl) or diaryl, arylalkyl, arylalkyloxy, alkenyl, alkynyl, cycloalkyl, cycloalkenyl, cycloalkylalkyl, cycloalkylalkyloxy, optionally substituted amino, hydroxy, hydroxyalkyl, acyl, oxo, alkanoyl, heteroaryl, heteroaryloxy, cycloheteroalkyl, arylheteroaryl, arylalkoxycarbonyl, heteroarylalkyl, heteroarylalkoxy, aryloxyalkyl, aryloxyaryl, alkylamido, alkanoylamino, arylcarbonylamino, alkoxycarbonyl, alkylaminocarbonyl, nitro, cyano, thiol, haloalkyl, trihaloalkyl, alkylthio or carboxyl(or alkyl ester thereof).

Unless otherwise indicated, the term “cycloalkyl” as employed herein alone or as part of another group includes saturated cyclic hydrocarbon groups or partially unsaturated (containing 1 or 2 double bonds) cyclic hydrocarbon groups, containing one ring and a total of 3 to 8 carbons, preferably 3 to 6 carbons, forming the ring As defined and claimed herein, the term “cycloalkyl” includes cycloalkyl groups as defined above optionally substituted with 1 or more substituents, such as those defined for alkyl.

The term “aryl” or “Ar” as employed herein alone or as part of another group refers to monocyclic and bicyclic aromatic groups containing 6 to 10 carbons in the ring portion (such as phenyl or naphthyl including 1-naphthyl and 2-naphthyl). As defined and claimed herein, the term “aryl” includes aryl groups as defined above optionally substituted through any available carbon atom(s) with 1 or more substitutents, such as halo, alkyl, haloalkyl, alkoxy, haloalkoxy, alkenyl, trifluoromethyl, trifluoromethoxy, alkynyl, hydroxy, amino, nitro, cyano, carboxyl(or alkyl ester thereof) or any of the other substituents described for alkyl.

Unless otherwise indicated, the term “alkenyl” as used herein by itself or as part of another group refers to straight or branched chain radicals of 2 to 20 carbons, preferably 2 to 12 carbons, and more preferably 2 to 8 carbons in the normal chain, which include one or more double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like. As defined and claimed herein, the term “alkenyl” includes alkenyl groups as defined above optionally substituted through any available carbon atom(s) with 1 or more substitutents, such as any of the substituents described for alkyl or aryl.

Unless otherwise indicated, the term “alkynyl” as used herein by itself or as part of another group refers to straight or branched chain radicals of 2 to 20 carbons, preferably 2 to 12 carbons and more preferably 2 to 8 carbons in the normal chain, which include one or more triple bonds in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl,3-undecynyl, 4-dodecynyl and the like. As defined and claimed herein, the term “alkynyl” includes alkynyl groups as defined above optionally substituted through any available carbon atom(s) with 1 or more substitutents, such as any of the substituents described for alkyl or aryl.

The term “cycloalkenyl” as employed herein alone or as part of another group refers to cyclic hydrocarbons containing 3 to 12 carbons, preferably 5 to 10 carbons and 1 or 2 double bonds. Exemplary cycloalkenyl groups include cyclopentenyl, cyclohexenyl, cyclohexadienyl, and cycloheptadienyl, which may be optionally substituted as defined for cycloalkyl. As defined and claimed herein, the term “cycloalkenyl” includes cycloalkenyl groups as defined above optionally substituted through any available carbon atom(s) with 1 or more substitutents, such as any of the substituents described for alkyl or aryl.

Unless otherwise indicated, the term “heteroaryl” or “heteroaromatic” as used herein alone or as part of another group refers to a 5- or 6-membered aromatic ring which includes 1, 2, 3 or 4 hetero atoms such as nitrogen, oxygen, or sulfur, and such rings fused to an aryl, cycloalkyl, heteroaryl or cycloheteroalkyl ring (e.g. benzothiophenyl, indole), and includes possible N-oxides. A “substituted heteroaryl” group includes a heteroaryl optionally substituted with one or more substituents such as any of the alkyl or aryl substituents set out above. As defined and claimed herein, the term “heteroaryl” includes heteroaryl groups as defined above optionally substituted through any available carbon atom(s) with 1 or more substitutents, such as any of the substituents described for alkyl or aryl.

The term “halogen” or “halo” as used herein alone or as part of another group refers to chlorine, bromine, fluorine, and iodine as well as CF₃, with chlorine or bromine being preferred.

The term “alkanoyl” as employed herein alone or as part of another group is alkyl linked to a carbonyl group.

The term “aroyl” as employed herein alone or as part of another group is aryl linked to a carbonyl group.

The term “tetrazole” as used herein is defined as having the structure:

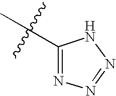

Unless otherwise indicated, the terms “alkoxy”, “aryloxy” or “heteroaryloxy” as employed herein alone or as part of another group includes any of the above alkyl, aryl or heteroaryl groups linked thorough an oxygen atom.

The term “cyano,” as used herein, refers to a —CN group.

The term “arylalkyl” and “heteroarylalkyl” as employed herein alone or as part of another group refer to alkyl groups as described above having an aryl or heteroaryl substituent. Representative examples of arylalkyl include, but are not limited to, benzyl, 2-phenylethyl, 3-phenylpropyl.

Unless otherwise indicated, the terms “arylalkoxy” and “cycloalkoxy” as employed herein alone or as part of another group include and aryl cycloalkyl groups linked thorough an oxygen atom.

The term “carboxylic acid” or “carboxyl”, as used herein, refers to a —COOH group.

The term “benzyl” as used herein refers to —CH₂C₆H₅, which may optionally be substituted as defined above for alkyl.

The compounds of formula I can be present as salts, in particular pharmaceutically acceptable salts. The compounds of formula I containing at least one acid group (for example COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono, di or tri-lower alkylamine, for example ethyl, tertbutyl, diethyl, diisopropyl, triethyl, tributyl or dimethyl-propylamine, or a mono, di or trihydroxy lower alkylamine, for example mono, di or triethanolamine. Corresponding internal salts may furthermore be formed. Salts which are unsuitable for pharmaceutical uses but which can be employed, for example, for the isolation or purification of free compounds I or their pharmaceutically acceptable salts, are also included.

Preferred salts of the compounds of formula I which include an acid group include sodium, potassium and magnesium salts and pharmaceutically acceptable organic amines.

The compounds of formula I may also have prodrug forms. Any compound that will be converted in vivo to provide the bioactive agent (i.e., the compound of formula I) is a prodrug within the scope and spirit of the invention.

Various forms of prodrugs are well known in the art. A comprehensive description of prodrugs and prodrug derivatives may be found in:

a.) The Practice of Medicinal Chemistry, Camille G. Wermuth et al., Ch 31, (Academic Press, 1996);

b.) Design of Prodrugs, edited by H. Bundgaard, (Elsevier, 1985); and

c.) A Textbook of Drug Design and Development, P. Krogsgaard-Larson and H. Bundgaard, eds. Ch 5, pgs 113-191 (Harwood Academic Publishers, 1991).

Preferred prodrugs include alkyl esters such as ethyl ester, or acyloxyalkyl esters such as pivaloyloxymethyl (POM).

All stereoisomers of the compounds of the instant invention are contemplated, either in admixture or in pure or substantially pure form. The compounds of the present invention can have asymmetric centers at any of the carbon atoms including any one or the R substituents. Consequently, compounds of formula I can exist in enantiomeric or diastereomeric forms or in mixtures thereof. The processes for preparation can utilize racemates, enantiomers or diastereomers as starting materials. When diastereomeric or enantiomeric products are prepared, they can be separated by conventional methods for example, chromatographic or fractional crystallization.

An administration of a therapeutic agent of the invention includes administration of a therapeutically effective amount of the agent of the invention. The term “therapeutically effective amount” as used herein refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The precise effective amount for a subject will depend upon the subject's size and health, the nature and extent of the condition being treated, recommendations of the treating physician, and the therapeutics or combination of therapeutics selected for administration. Thus, it is not useful to specify an exact effective amount in advance.

The compounds of formula I may be prepared by the exemplary processes described in the following reaction schemes, as well as relevant published literature procedures that are used by one skilled in the art. Exemplary reagents and procedures for these reactions appear hereinafter and in the working Examples. Protection and deprotection in the Schemes below may be carried out by procedures generally known in the art (see, for example, T. W. Greene & P. G. M. Wuts, “Protecting Groups in Organic Synthesis”, 3^{rd} Edition, Wiley, 1999).

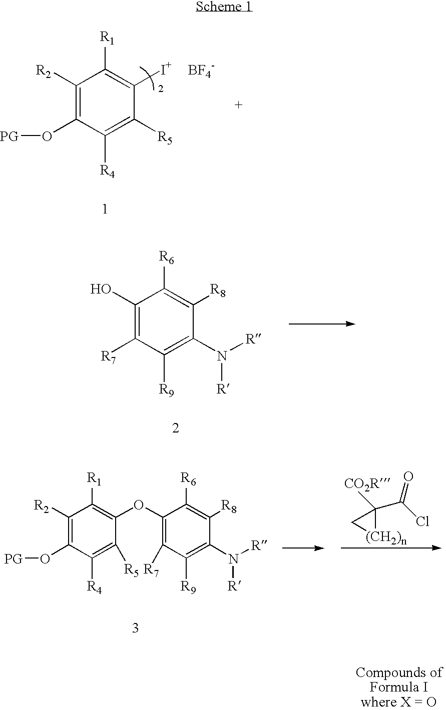

Scheme 1 depicts a general synthetic approach to compounds of formula I for which X=O that utilizes the coupling of an appropriately substituted iodonium salt 1 to the appropriate phenol 2 to provide intermediate 3. In structure 1 and all other applicable structures contained in further schemes described below, PG refers to a protecting group appropriate for the functional group indicated (in this instance, for a phenolic oxygen). The specific protecting groups for each particular intermediate are well understood by those versed in the art (see also the reference, “Protecting Groups in Organic Synthesis”, cited above). Subsequent protecting group and functional group manipulation provides the desired compounds of formula I. For example, intermediate 2 may be a nitrophenol (R′ and R″ are oxygen) and the resulting coupling product would be the corresponding diaryl ether nitro compound 3 where R′=R″=O. This nitro intermediate can be readily reduced to the corresponding aryl amine (see discussion below). The resulting aryl amine can then be readily acylated to provide the desired compounds of formula I (X=O). Intermediate 2 may also be a protected amino function, for example R′=R₅ and R″=PG. The protecting group (PG) may be carbamates such as t-butyloxycarbonyl (BOC) or benzyloxycarbonyl (CBZ), which may be later removed by acidolysis and/or hydrogenolysis under standard conditions. Acylation of the resulting aryl amine, again by means well-known to those versed in the art, provides the desired compounds of formula I. In addition, the aryl amine (intermediate 3 where R′=R″=H) resulting from reduction of a nitrobenzene coupling product can be reacted with an aldehyde in a reductive amination reaction, thus installing the group R₅ which comes from the aldehyde moiety. Reductive amination procedures, such as by the use of sodium cyanoborohydride or sodium triacetoxyborohydride, are well known to those skilled in the art. The resulting product can then be acylated by standard procedures to provide compounds of formula I.

The iodonium salt methodology depicted in Scheme 1 is amply described in the literature for the synthesis of thyroid hormone analogs (“Novel Thyroid Receptor Ligands and Methods, Y.-L. Li, Y. Liu, A. Hedfors, J. Malm, C. Mellin, M. Zhang, PCT Int. App. WO 9900353 A1 990107; D. M. B. Hickey et al., J. Chem. Soc. Perkin Trans. I, 3103-3111, 1988; N. Yokoyama et al., J. Med. Chem., 38, 695-707, 1995), and to diaryl ethers in general (E. A. Couladouros, V. I. Moutsos, Tetrahedron Lett., 40, 7023-7026, 1999).

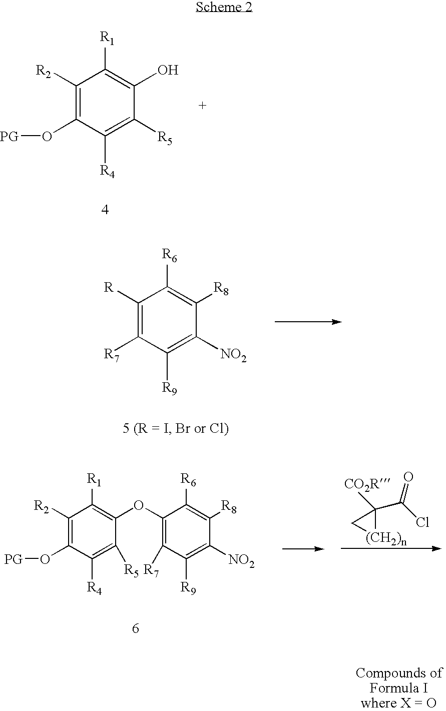

Scheme 2 depicts another general synthetic approach to compounds of formula I for which X=O in which an appropriately substituted nitrobenzene intermediate 5 is condensed with an appropriately substituted phenol 4 to provide the nitro intermediate 6. The nitro function in intermediate 6 can be reduced to an amino group by methods well known in the art, such as the use of catalytic hydrogenation in the presence of, for example, Raney nickel or palladium on charcoal catalyst, in a polar solvent such as glacial acetic acid or ethanol. Alternatively, the reduction can be accomplished using iron powder in aqueous glacial acetic acid at ambient temperatures. Subsequent protecting group and functional group manipulation provides the desired compounds of formula I.

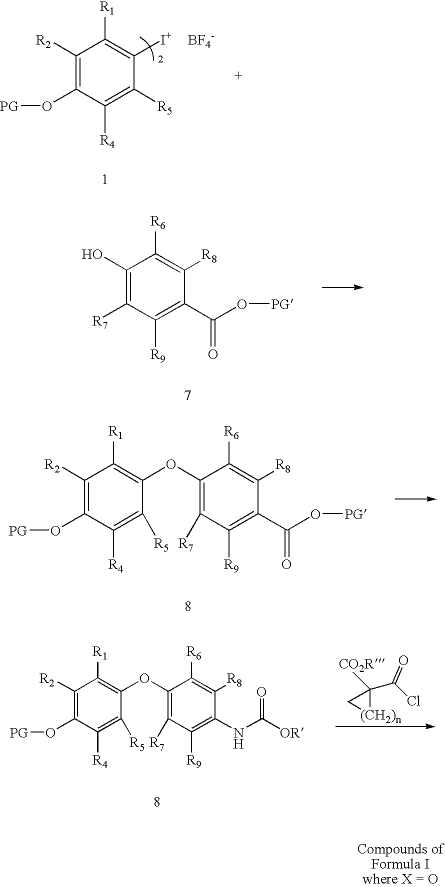

Another general approach to the synthesis of compounds of formula I in which X=O is shown in Scheme 3. In this approach, an appropriately substituted iodonium salt 1 is coupled to the appropriately substituted 4-hydroxybenzoic acid intermediate 7. The carboxyl protecting group (PG′) in the resulting coupling product 8 is then removed. The resulting free carboxylic acid intermediate corresponding to 8 is then subjected to a Curtius rearrangement by the use of known reagents for that transformation such as diphenylphosphoryl azide (DPPA). The Curtius rearrangement intermediate can be trapped by either t-butanol or benzyl alcohol to give the product 9, a t-butyloxycarbonyl (BOC) or a benzyloxycarbonyl (CBZ) protected aniline, respectively. These protecting groups can be removed by methods well known in the art to give the corresponding free amine group. The amine can then be acylated to give compounds of formula I with X=O by one of any number of well-established procedures, such as acylation with a free carboxylic acid by using a coupling reagent such as dicyclohexyl carbodiimide (DCC) or (1-[3-(dimethylamino)propyl]-3-ethylcarbodiimide (EDCI). Alternatively, the free amine can be acylated using a carboxylic acid chloride derivative in the presence of an equivalent amount of a tertiary organic amine such as triethylamine or N-methyl morpholine.

With reference to the syntheses described above, the general synthesis of diaryl ethers for thyromimetics is well precedented in the literature (P. D. Leeson, J. C. Emmett, J. Chem. Perkin Trans. I, 3085-3096, 1988; N. Yokoyama et al., J. Med. Chem., 38, 695-707, 1995).

Methods applicable to the synthesis of compounds of formula I in which X=O and R₆ and R₇ are independently varied as hydrogen, halogen and alkyl are described in “Novel Thyroid Receptor Ligands and Methods, Y.-L. Li, Y. Liu, A. Hedfors, J. Malm, C. Mellin, M. Zhang, PCT Int. App. WO 9900353 A1 990107.

Further means for synthesizing compounds of formula I in which X=O, NH, S, CO or CH₂ are generally described in the literature (for X=O: D. M. B. Hickey et al., J. Chem. Soc. Perkin Trans. I, 3097-3102, 1988; Z.-W. Guo et al., J. Org. Chem., 62, 6700-6701, 1997; D. M. T. Chan et al., Tetrahedron Lett., 39, 2933-2936, 1998; D. A. Evans et al., Tetrahedron Lett., 39, 2937-2940, 1998; G. M. Salamonczyk et al., Tetrahedron Lett., 38, 6965-6968, 1997; J.-F. Marcoux, J. Am. Chem. Soc., 119, 10539-10540, 1997; A. V. Kalinin et al., J. Org. Chem., 64, 2986-2987, 1999; for X=N: D. M. T. Chan et al., Tetrahedron Lett., 39, 2933-2936, 1998; J. P. Wolfe et al., J. Am. Chem. Soc., 118, 7215, 1996; M. S. Driver, J. F. Hartwig, J. Am. Chem. Soc., 118, 7217, 1996; see references in the review by C. G. Frost, P. Mendonca, J. Chem. Soc. Perkin I, 2615-2623, 1998; for X=S: C. R. Harrington, Biochem. J., 43, 434-437, 1948; A. Dibbo et al., J. Chem. Soc., 2890-2902, 1961; N. Yokoyama et al., U.S. Pat. No. 5,401,772, 1995; for X=CO or CH₂: L. Horner, H. H. G. Medem, Chem. Ber., 85, 520-530, 1952; G. Chiellini et al., Chemistry & Biology, 5, 299-306, 1998).

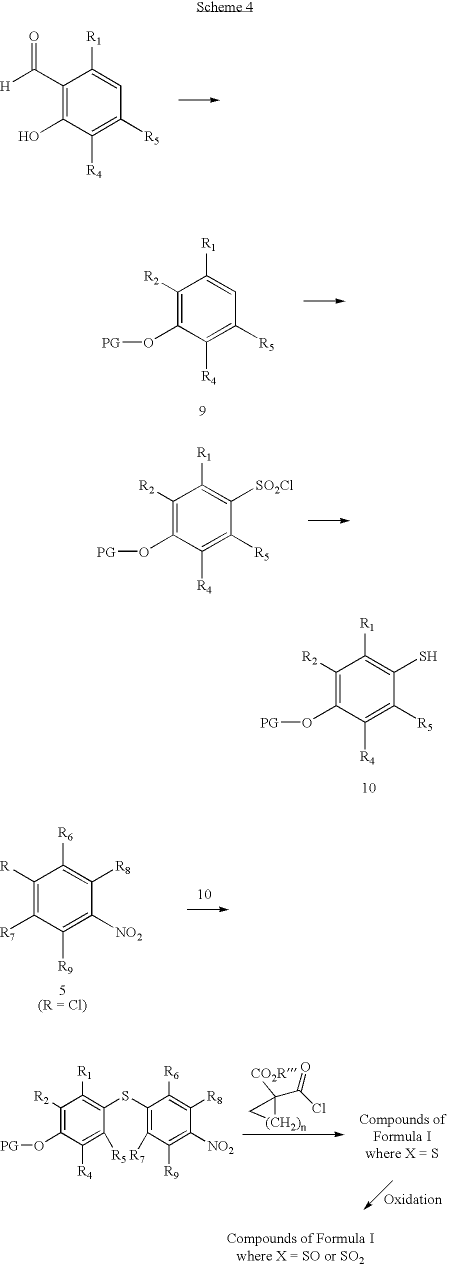

Compounds of formula I where X is S, SO or SO₂ can be prepared as outlined in Scheme 4. Beginning with the appropriate phenolic ether 9, chlorosulfonolation with chlorosulfonic acid in a solvent such as CH₂Cl₂ followed by reduction with a metal such Zn in aq. H₂SO₄ or AcOH generates the aryl thiol 10. The aryl thiol intermediate 10 can be coupled with aryl halides of structure 5, then reduced, acylated and deprotected to generate compounds of Formula I where X is S. Compounds of Formula I where X is SO or SO₂ can be prepared in a similar manner except that prior to deprotection the sulfur is oxidized to the appropriate oxidation state using m-chloroperbenzoic acid. The phenolic ether 9 described above are either commercially available, or in the case where R₂ is iPr, readily prepared following the procedure described in R. M. Jones et al, J. Org. Chem., 2001, 66, 3435-3441 via sequential treatment of the appropriate substituted salicylaldehyde with BOC anhydride and excess alkyl lithium.

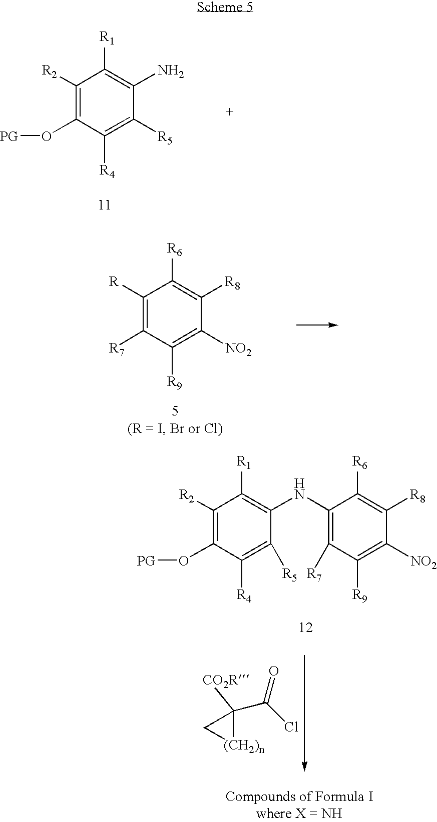

In a similar fashion (Scheme 5) compounds of Formula I where X is NH can be prepared by nitration of intermediate 9 (from Scheme 4), reduction to the aniline 11, followed by coupling with 5 to generate the desired diaryl amine 12. Anilines represented by 12 can be converted to compounds of Formula I where X is NH following reduction, acylation and deprotection.

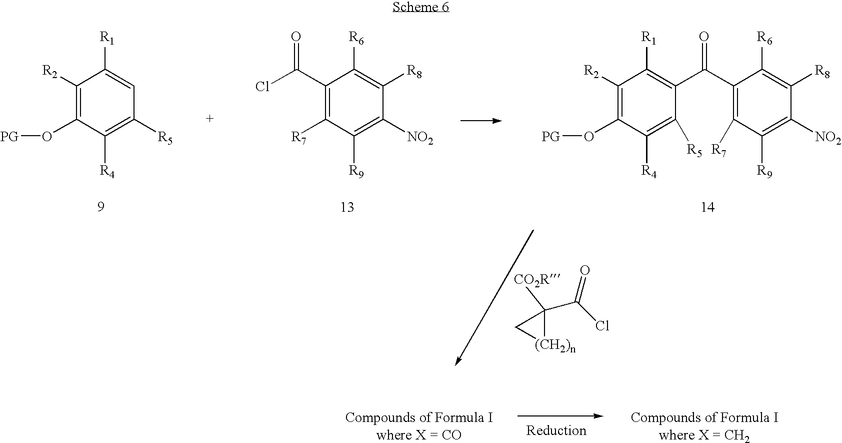

Compounds of formula I where X is CO or CH₂ (Scheme 6) can be prepared by acylation of compound 9 with an acid chloride, such as 13, in the presence of a Lewis acid catalyst, such AlCl₃ in a solvent, such as CS₂ or CH₂Cl₂₁ to generate the prerequisite ketone 14. Ketones represented by 14 can be converted to compounds of formula I where X is CO following Fe mediated reduction of the NO₂ group, acylation and deprotection. Subsequent reduction of the ketone carbonyl with Et₃SiH/BF₃.Et₂O generates compounds of formula I where X is CH₂.

### Utilites & Combinations

A. Utilities

The compounds of the present invention are thyroid receptor ligands, and include compounds which are, for example, selective agonists, partial agonists, antagonists or partial antagonists of the thyroid receptor. Preferably compounds of the present invention possess activity as agonists of the thyroid receptor, and may be used in the treatment of diseases or disorders associated with thyroid receptor activity. In particular, compounds of the present invention may be used in the treatment of diseases or disorders associated with metabolic dysfunction or which are dependent upon the expression of a T₃ regulated gene.

Accordingly, the compounds of the present invention can be administered to mammals, preferably humans, for the treatment of a variety of conditions and disorders, including, but not limited to hypothyroidism; subclinical hyperthyroidism; non-toxic goiter; atherosclerosis; thyroid hormone replacement therapy (e.g., in the elderly); malignant tumor cells containing the thyroid receptor; papillary or follicular cancer; maintenance of muscle strength and function (e.g., in the elderly); reversal or prevention of frailty or age-related functional decline (“ARFD”) in the elderly (e.g., sarcopenia); treatment of catabolic side effects of glucocorticoids; prevention and/or treatment of reduced bone mass, density or growth (e.g., osteoporosis and osteopenia); treatment of chronic fatigue syndrome (CFS); accelerating healing of complicated fractures, e.g. distraction osteogenesis; in joint replacement; eating disorders (e.g., anorexia); treatment of obesity and growth retardation associated with obesity; treatment of depression, nervousness, irritability and stress; treatment of reduced mental energy and low self-esteem (e.g., motivation/assertiveness); improvement of cognitive function (e.g., the treatment of dementia, including Alzheimer's disease and short term memory loss); treatment of catabolism in connection with pulmonary dysfunction and ventilator dependency; treatment of cardiac dysfunction (e.g., associated with valvular disease, myocardial infarction, cardiac hypertrophy or congestive heart failure); lowering blood pressure; protection against ventricular dysfunction or prevention of reperfusion events; treatment of hyperinsulinemia; stimulation of osteoblasts, bone remodeling and cartilage growth; regulation of food intake; treatment of insulin resistance, including NIDDM, in mammals (e.g., humans); treatment of insulin resistance in the heart; treatment of congestive heart failure; treatment of musculoskeletal impairment (e.g., in the elderly); improvement of the overall pulmonary function; skin disorders or diseases, such as glucocorticoid induced dermal atrophy, including restoration of dermal atrophy induced by topical glucocorticoids, and the prevention of dermal atrophy induced by topical glucocorticoids (such as the simultaneous treatment with topical glucocorticoid or a pharmacological product including both glucocorticoid and a compound of the invention), the restoration/prevention of dermal atrophy induced by systemic treatment with glucocorticoids, restoration/prevention of atrophy in the respiratory system induced by local treatment with glucocorticoids, UV-induced dermal atrophy, dermal atrophy induced by aging (wrinkles, etc.), wound healing, keloids, stria, cellulite, roughened skin, actinic skin damage, lichen planus, ichtyosis, acne, psoriasis, Dernier's disease, eczema, atopic dermatitis, chloracne, pityriasis and skin scarring.

The term treatment is also intended to include prophylactic treatment.

In addition, the conditions, diseases, and maladies collectively referenced to as “Syndrome X” or Metabolic Syndrome as detailed in Johannsson J. Clin. Endocrinol. Metab., 82, 727-34 (1997), may be treated employing the compounds of the invention.

B. Combinations

The present invention includes within its scope pharmaceutical compositions comprising, as an active ingredient, a therapeutically effective amount of at least one of the compounds of formula I, alone or in combination with a pharmaceutical carrier or diluent. Optionally, compounds of the present invention can be used alone, in combination with other compounds of the invention, or in combination with one or more other therapeutic agent(s), e.g., an antidiabetic agent or other pharmaceutically active material.

The compounds of the present invention may be employed in combination with other modulators and/or ligands of the thyroid receptor or other suitable therapeutic agents useful in the treatment of the aforementioned disorders including: anti-diabetic agents; anti-osteoporosis agents; anti-obesity agents; growth promoting agents (including growth hormone secretagogues); anti-inflammatory agents; anti-anxiety agents; anti-depressants; anti-hypertensive agents; cardiac glycosides; cholesterol/lipid lowering agents; appetite suppressants; bone resorption inhibitors; thyroid mimetics (including other thyroid receptor agonists); anabolic agents; and anti-tumor agents.

Examples of suitable anti-diabetic agents for use in combination with the compounds of the present invention include biguanides (e.g., metformin or phenformin), glucosidase inhibitors (e.g,. acarbose or miglitol), insulins (including insulin secretagogues or insulin sensitizers), meglitinides (e.g., repaglinide), sulfonylureas (e.g., glimepiride, glyburide, gliclazide, chlorpropamide and glipizide), biguanide/glyburide combinations (e.g., Glucovance®), thiazolidinediones (e.g., troglitazone, rosiglitazone and pioglitazone), PPAR-alpha agonists, PPAR-gamma agonists, PPAR alpha/gamma dual agonists, SGLT2 inhibitors, glycogen phosphorylase inhibitors, inhibitors of fatty acid binding protein (aP2), glucagon-like peptide-1 (GLP-1), and dipeptidyl peptidase IV (DP4) inhibitors.

Examples of suitable anti-osteoporosis agents for use in combination with the compounds of the present invention include alendronate, risedronate, PTH, PTH fragment, raloxifene, calcitonin, RANK ligand antagonists, calcium sensing receptor antagonists, TRAP inhibitors, selective estrogen receptor modulators (SERM) and AP-1 inhibitors.

Examples of suitable anti-obesity agents for use in combination with the compounds of the present invention include aP2 inhibitors, PPAR gamma antagonists, PPAR delta agonists, beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), other thyroid receptor beta drugs, such as a thyroid receptor ligand as disclosed in WO 97/21993 (U. Cal SF), WO 99/00353 (KaroBio) and GB98/284425 (KaroBio), a cannabinoid-1 receptor antagonist, such as SR-141716 (Sanofi) and/or an anorectic agent, such as dexamphetamine, phentermine, phenylpropanolamine or mazindol.

The compounds of the present invention may be combined with growth promoting agents, such as, but not limited to, TRH, diethylstilbesterol, theophylline, enkephalins, E series prostaglandins, compounds disclosed in U.S. Pat. No. 3,239,345, e.g., zeranol, and compounds disclosed in U.S. Pat. No. 4,036,979, e.g., sulbenox or peptides disclosed in U.S. Pat. No. 4,411,890.

The compounds of the invention may also be used in combination with growth hormone secretagogues such as GHRP-6, GHRP-1 (as described in U.S. Pat. No. 4,411,890 and publications WO 89/07110 and WO 89/07111), GHRP-2 (as described in WO 93/04081), NN703 (Novo Nordisk), LY444711 (Lilly), MK-677 (Merck), CP424391 (Pfizer) and B-HT920, or with growth hormone releasing factor and its analogs or growth hormone and its analogs or somatomedins including IGF-1 and IGF-2, or with alpha-adrenergic agonists, such as clonidine or serotinin 5-HTD agonists, such as sumatriptan, or agents which inhibit somatostatin or its release, such as physostigmine and pyridostigmine. A still further use of the disclosed compounds of the invention is in combination with parathyroid hormone, PTH(1-34) or bisphosphonates, such as MK-217 (alendronate).

A still further use of the compounds of the invention is in combination with estrogen, testosterone, a selective estrogen receptor modulator, such as tamoxifen or raloxifene, or other androgen receptor modulators, such as those disclosed in Edwards, J. P. et al., Bio. Med. Chem. Let., 9, 1003-1008 (1999) and Hamann, L. G. et al., J. Med. Chem., 42, 210-212 (1999).

A further use of the compounds of this invention is in combination with steriodal or non-steroidal progesterone receptor agonists (“PRA”), such as levonorgestrel, medroxyprogesterone acetate (MPA).

Examples of suitable anti-inflammatory agents for use in combination with the compounds of the present invention include prednisone, dexamethasone, Enbrel®, cyclooxygenase inhibitors (i.e., COX-1 and/or COX-2 inhibitors such as NSAIDs, aspirin, indomethacin, ibuprofen, piroxicam, Naproxen®, Celebrex®, Vioxx®), CTLA4-Ig agonists/antagonists, CD40 ligand antagonists, IMPDH inhibitors, such as mycophenolate (CellCept®), integrin antagonists, alpha-4 beta-7 integrin antagonists, cell adhesion inhibitors, interferon gamma antagonists, ICAM-1, tumor necrosis factor (TNF) antagonists (e.g., infliximab, OR1384), prostaglandin synthesis inhibitors, budesonide, clofazimine, CNI-1493, CD4 antagonists (e.g., priliximab), p38 mitogen-activated protein kinase inhibitors, protein tyrosine kinase (PTK) inhibitors, IKK inhibitors, and therapies for the treatment of irritable bowel syndrome (e.g., Zelmac® and Maxi-K® openers such as those disclosed in U.S. Pat. No. 6,184,231 B1).

Example of suitable anti-anxiety agents for use in combination with the compounds of the present invention include diazepam, lorazepam, buspirone, oxazepam, and hydroxyzine pamoate.

Examples of suitable anti-depressants for use in combination with the compounds of the present invention include citalopram, fluoxetine, nefazodone, sertraline, and paroxetine.

For the treatment of skin disorders or diseases as described above, the compounds of the present invention may be used alone or optionally in combination with a retinoid, such as tretinoin, or a vitamin D analog.

Examples of suitable anti-hypertensive agents for use in combination with the compounds of the present invention include beta adrenergic blockers, calcium channel blockers (L-type and T-type; e.g. diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable cardiac glycosides for use in combination with the compounds of the present invention include digitalis and ouabain.

Examples of suitable cholesterol/lipid lowering agents for use in combination with the compounds of the present invention include HMG-COA reductase inhibitors, squalene synthetase inhibitors, fibrates, bile acid sequestrants, ACAT inhibitors, MTP inhibitors, lipooxygenase inhibitors, an ileal Na⁺/bile acid cotransporter inhibitor, cholesterol absorption inhibitors, and cholesterol ester transfer protein inhibitors (e.g., CP-529414).

MTP inhibitors which may be employed herein in combination with one or more compounds of formula I include MTP inhibitors as disclosed in U.S. Pat. No. 5,595,872, U.S. Pat. No. 5,739,135, U.S. Pat. No. 5,712,279, U.S. Pat. No. 5,760,246, U.S. Pat. No. 5,827,875, U.S. Pat. No. 5,885,983 and U.S. Pat. No. 5,962,440 all incorporated herein by reference.

The HMG CoA reductase inhibitors which may be employed in combination with one or more compounds of formula I include mevastatin and related compounds as disclosed in U.S. Pat. No. 3,983,140, lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Further HMG CoA reductase inhibitors which may be employed herein include fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pyrazole analogs of mevalonolactone derivatives as disclosed in U.S. Pat. No. 4,613,610, indene analogs of mevalonolactone derivatives, as disclosed in PCT application WO 86/03488, 6-[2-(substituted-pyrrol-1-yl)-alkyl)pyran-2-ones and derivatives thereof, as disclosed in U.S. Pat. No. 4,647,576, Searle's SC-45355 (a 3-substituted pentanedioic acid derivative) dichloroacetate, imidazole analogs of mevalonolactone, as disclosed in PCT application WO 86/07054, 3-carboxy-2-hydroxy-propane-phosphonic acid derivatives, as disclosed in French Patent No. 2,596,393, 2,3-disubstituted pyrrole, furan and thiophene derivatives, as disclosed in European Patent Application No. 0221025, naphthyl analogs of mevalonolactone, as disclosed in U.S. Pat. No. 4,686,237, octahydronaphthalenes, such as disclosed in U.S. Pat. No. 4,499,289, keto analogs of mevinolin (lovastatin), as disclosed in European Patent Application No.0,142,146 A2, as well as other known HMG CoA reductase inhibitors.

The squalene synthetase inhibitors which may be used in combination with the compounds of the present invention include, but are not limited to, α-phosphono-sulfonates disclosed in U.S. Pat. No. 5,712,396, those disclosed by Biller et al, J. Med. Chem., 1988, Vol. 31, No. 10, pp 1869-1871, including isoprenoid (phosphinylmethyl)phosphonates, terpenoid pyrophosphates disclosed by P. Ortiz de Montellano et al, J. Med. Chem., 1977, 20, 243-249, the farnesyl diphosphate analog A and presqualene pyrophosphate (PSQ-PP) analogs as disclosed by Corey and Volante, J. Am. Chem. Soc., 1976, 98, 1291-1293, phosphinylphosphonates reported by McClard, R. W. et al, J.A.C.S., 1987, 109, 5544 and cyclopropanes reported by Capson, T. L., PhD dissertation, June, 1987, Dept. Med. Chem. U of Utah, Abstract, Table of Contents, pp 16, 17, 40-43, 48-51, as well as other squalene synthetase inhibitors as disclosed in U.S. Pat. No. 4,871,721 and 4,924,024 and in Biller, S. A., Neuenschwander, K., Ponpipom, M. M., and Poulter, C. D., Current Pharmaceutical Design, 2, 1-40 (1996).

Bile acid sequestrants which may be used in combination with the compounds of the present invention include cholestyramine, colestipol and DEAE-Sephadex (Secholex®, Policexide®), as well as lipostabil (Rhone-Poulenc), Eisai E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid, acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

ACAT inhibitors suitable for use in combination with compounds of the invention include ACAT inhibitors as described in, Drugs of the Future 24, 9-15 (1999), (Avasimibe); “The ACAT inhibitor, Cl-1011 is effective in the prevention and regression of aortic fatty streak area in hamsters”, Nicolosi et al, Atherosclerosis (Shannon, Irel). (1998), 137(1), 77-85; “The pharmacological profile of FCE 27677: a novel ACAT inhibitor with potent hypolipidemic activity mediated by selective suppression of the hepatic secretion of ApoB100-containing lipoprotein”, Ghiselli, Giancarlo, Cardiovasc. Drug Rev. (1998), 16(1), 16-30; “RP 73163: a bioavailable alkylsulfinyl-diphenylimidazole ACAT inhibitor”, Smith, C., et al, Bioorg. Med. Chem. Lett. (1996), 6(1), 47-50; “ACAT inhibitors: physiologic mechanisms for hypolipidemic and anti-atherosclerotic activities in experimental animals”, Krause et al, Editor(s): Ruffolo, Robert R., Jr.; Hollinger, Mannfred A., Inflammation: Mediators Pathways (1995), 173-98, Publisher: CRC, Boca Raton, Fla.; “ACAT inhibitors: potential anti-atherosclerotic agents”, Sliskovic et al, Curr. Med. Chem. (1994), 1(3), 204-25; “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)methyl]ureas with enhanced hypocholesterolemic activity”, Stout et al, Chemtracts: Org. Chem. (1995), 8(6), 359-62.

Examples of suitable cholesterol absorption inhibitor for use in combination with the compounds of the invention include SCH48461 (Schering-Plough), as well as those disclosed in Atherosclerosis 115, 45-63 (1995) and J. Med. Chem. 41, 973 (1998).

Examples of suitable ileal Na⁺/bile acid cotransporter inhibitors for use in combination with the compounds of the invention include compounds as disclosed in Drugs of the Future, 24, 425-430 (1999).

Examples of suitable thyroid mimetics for use in combination with the compounds of the present invention include thyrotropin, polythyroid, KB-130015, and dronedarone.

Examples of suitable anabolic agents for use in combination with the compounds of the present invention include testosterone, TRH diethylstilbesterol, estrogens, 5-agonists, theophylline, anabolic steroids, dehydroepiandrosterone, enkephalins, E-series prostagladins, retinoic acid and compounds as disclosed in U.S. Pat. No. 3,239,345, e.g., Zeranol®; U.S. Pat. No. 4,036,979, e.g., Sulbenox® or peptides as disclosed in U.S. Pat. No. 4,411,890.

The aforementioned patents and patent applications are incorporated herein by reference.

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with a hypolypidemic agent, an antidepressant, a bone resorption inhibitor and/or an appetite suppressant, the compounds of formula I may be employed in a weight ratio to the additional agent within the range from about 500:1 to about 0.005:1, preferably from about 300:1 to about 0.01:1.

Where the antidiabetic agent is a biguanide, the compounds of formula I may be employed in a weight ratio to biguanide within the range from about 0.01:1 to about 100:1, preferably from about 0.5:1 to about 2:1.

The compounds of formula I may be employed in a weight ratio to a glucosidase inhibitor within the range from about 0.01:1 to about 100:1, preferably from about 0.5:1 to about 50:1.

The compounds of formula I may be employed in a weight ratio to a sulfonylurea in the range from about 0.01:1 to about 100:1, preferably from about 0.2:1 to about 10:1.

The compounds of formula I may be employed in a weight ratio to a thiazolidinedione in an amount within the range from about 0.01:1 to about 100:1, preferably from about 0.5:1 to about 5:1.

The thiazolidinedione may be employed in amounts within the range from about 0.01 to about 2000 mg/day, which may optionally be administered in single or divided doses of one to four times per day.

Further, where the sulfonylurea and thiazolidinedione are to be administered orally in an amount of less than about 150 mg, these additional agents may be incorporated into a combined single tablet with a therapeutically effective amount of the compounds of formula I.

Metformin, or salt thereof, may be employed with the compounds of formula I in amounts within the range from about 500 to about 2000 mg per day, which may be administered in single or divided doses one to four times daily.

The compounds of formula I may be employed in a weight ratio to a PPAR-alpha agonist, a PPAR-gamma agonist, a PPAR-alpha/gamma dual agonist, an SGLT2 inhibitor and/or an aP2 inhibitor within the range from about 0.01:1 to about 100:1, preferably from about 0.5:1 to about 5:1.

An MTP inhibitor may be administered orally with the compounds of formula I in an amount within the range of from about 0.01 mg/kg to about 100 mg/kg and preferably from about 0.1 mg/kg to about 75 mg/kg, one to four times daily.

A preferred oral dosage form, such as tablets or capsules, may contain the MTP inhibitor in an amount of from about 1 to about 500 mg, preferably from about 2 to about 400 mg, and more preferably from about 5 to about 250 mg, administered on a regimen of one to four times daily.

For parenteral administration, the MTP inhibitor may be employed in an amount within the range of from about 0.005 mg/kg to about 10 mg/kg and preferably from about 0.005 mg/kg to about 8 mg/kg, administered on a regimen of one to four times daily.

A HMG CoA reductase inhibitor may be administered orally with the compounds of formula I within the range of from about 1 to 2000 mg, and preferably from about 4 to about 200 mg.

A preferred oral dosage form, such as tablets or capsules, will contain the HMG CoA reductase inhibitor in an amount from about 0.1 to about 100 mg, preferably from about 5 to about 80 mg, and more preferably from about 10 to about 40 mg.

A squalene synthetase inhibitor may be administered with the compounds of formula I within the range of from about 10 mg to about 2000 mg and preferably from about 25 mg to about 200 mg.

A preferred oral dosage form, such as tablets or capsules, will contain the squalene synthetase inhibitor in an amount of from about 10 to about 500 mg, preferably from about 25 to about 200 mg.

The compounds of formula I of the invention can be administered orally or parenterally, such as subcutaneously or intravenously, as well as by nasal application, rectally or sublingually to various mammalian species known to be subject to such maladies, e.g., humans, in an effective amount within the dosage range of abut 0.01 μg/kg to about 1000 μg/kg, preferably about 0.1 μg/kg to 100 μg/kg, more preferably about 0.2 μg/kg to about 50 μg/kg (or form about 0.5 to 2500 mg, preferably from about 1 to 2000 mg) in a regimen of single, two or four divided daily doses.

The compounds of the formula I can be administered for any of the uses described herein by any suitable means, for example, orally, such as in the form of tablets, capsules, granules or powders; sublingually; bucally; parenterally, such as by subcutaneous, intravenous, intramuscular, or intrasternal injection or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories; in dosage unit formulations containing non-toxic, pharmaceutically acceptable vehicles or diluents. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate and/or lactose and/or other excipients, binders, extenders, disintegrants, diluents and lubricants such as those known in the art. The compounds of formula I can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use.

Exemplary compositions for nasal aerosol or inhalation administration include solutions in saline which can contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for rectal administration include suppositories which can contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

It will be understood that the specific dose level and frequency of dosage for any particular subject can be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

The following working examples serve to better illustrate, but not limit, some of the preferred embodiments of the present invention.

### EXAMPLE 1

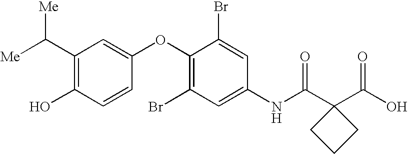

### N-[3,5-dibromo-4-[4-hydroxy-3-(1-methylethyl)-phenoxy]phenyl]-1-carbamylcyclobutane-1-carboxylic acid

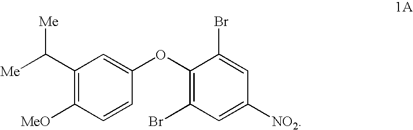

Bis(3-isopropyl-4-methoxyphenyl)iodonium tetrafluoroborate (32.8 g, 64 mmol)(see Yokayama et al, J. Med. Chem., 1995, 38, 695-707 for preparation), 2,6-dibromo-4-nitrophenol (12.6 g, 42 mmol), and Cu powder [Lancaster 300 mesh (6.8 g, 108 mmol)] were suspended in 400 ml of CH₂Cl₂ in a flask covered with aluminum foil. While stirring, Et₃N (18.4 mL, 219 mmol) was added and the reaction mixture was stirred under argon in the dark for 4 days. The crude reaction mixture was concentrated to about 70 mL and then chromatographed in two portions on Merck silica gel eluting with 3% to 5% EtOAc/hexanes. The combined yield of the 3-isopropyl-4-methoxy-2′,6′-dibromo-4′-nitrodiphenyl ether was 15.4 g (81.9%).

An alternative route synthesizing Compound 1A more amenable to scale-up entails the following conversions of 2-isopropylphenol to 3-isopropyl-4-methoxyphenol and 4-nitro-2,6-dibromophenol to the corresponding iodide whereupon these two entities were condensed to generate Compound 1A:

To a stirred 20° C. solution comprising KOH (1154 g, 4.75 mol) and Bu₄N⁺ HSO₄⁻ (140 g, 0.41 mol) in H₂O (5.6 L) was added commercially available 2-isopropylphenol (590 g, 4.33mol) in CH₂Cl₂ (5.6 L). After 30 min, MeI (741 g, 5.22 mol) was added prior to stirring the reaction overnight. After separation of the layers, Et₃N (185 mL, 1.3 mol) was added to the CH₂C12 fraction to destroy the residual MeI. After 15 min, the CH₂Cl₂ was removed under vacuum and the salts suspended in cyclohexane (4 L) prior to filtration. The cyclohexane filtrate was sequentially washed with 2N HCl followed by 2 brine washes. Concentration under vacuum yielded 2-isopropylanisole (612 g, 94%) as a light yellow oil.

To a stirred solution of 2-isopropylanisole (859 g, 5.85 mol) and POCl₃ (2690 g, 17.5 mol) at 80° C. under N₂, DMF (1584 mL, 20.46 mol) was slowly added at a rate such that the temperature remained between 80-90° C. After stirring for 16 hr at 85° C., the dark solution was poured cautiously onto 7 Kg of ice (Quench required 1.5 hr due to iolent exotherm). The mixture was extracted twice with EtOAc (total volume 16 L). The combined EtOAc layers were washed once with aq. NaHCO₃ and then with brine. Upon concentration, 881 g of 4-formyl-2-isopropylanisole was obtained.

To a solution of 4-formyl-2-isopropylanisole (880 g, 4.94 mol) in THF (4.56 L) and cyclohexane (3.74 L) at 20° C. was added a solution of NaHSO₃ (1.31 kg, 12.56 mol) in H₂O (4.36 L). After stirring overnight, the crystals were collected by filtration, washed with 3:1 cyclohexane/THF prior to drying under vacuum to yield 1.3 kg of bisulfite adduct. To a stirred solution of the dried adduct in 1:4 H₂O/MeOH (13 L) containing p-Tos.OH H₂O (908 g, 4.77 mol) was slowly added 30% H₂O₂ (1.625 L, 16.1 mol) over 1.75 hr at a rate such that the temperature remained between 0-5° C. After stirring overnight at 20° C., the reaction was monitored by HPLC. Additional H₂O₂ was added if starting material remained. Upon completion, the reaction was cooled to 4° C., whereupon a solution of Na₂SO₃ (1.86 kg, 10.68 mo) in 6.5 L of H₂O was added at a rate such that the temperature did not exceed 34° C. After stirring for 1 hr, the solids were filtered and washed with EtOAc. The aqueous layer was extracted with EtOAc. The combined EtOAc fractions were washed sequentially with aq. NaHCO₃ and brine. Upon concentration, 3-isopropyl-4-methoxyphenol (510 g, 67% conversion) was obtained in 93% purity.

To a stirred 0° C. solution of 2,6-dibromo-4-nitrophenol (0.8 kg, 2.69M) in CH₂Cl₂ (3.2 L) under N₂, was added pyridine (436 mL, 5.39 mol). After stirring for 5 min at 0° C., triflic anhydride (544 mL, 3.23 mol) in CH₂Cl₂ (400 mL) was added at a rate to maintain a temperature <10° C. Upon completion of the addition, the reaction was stirred approx. 30 min at 20° C. until deemed complete by HPLC. The reaction was quenched by dropwise addition of 1N HCl (1.6 L), such that the temperature remained <35° C. After separation of the layers, the CH₂Cl₂ layer was washed with sat'd NaHCO₃, then brine. Concentration yielded 2,6-dibromo-4-nitrophenyl triflate (1140 g, 99%) after drying overnight at 20° C. under vacuum.

A solution of 2,6-dibromo-4-nitrophenyl triflate (46.7 g, 109 mmol) and NaI (65.3 g, 436 mmol) were heated to 100° C. under Ar in DMF (121 mL) for 20 hr. After cooling, the slurry was transferred into ice (H₂O). After stirring for 10 min, the suspension was filtered. The filter cake washed with H₂O and air dried to yield 30.5 g (69%) of 3,5-dibromo-4-iodonitrobenzene as a brownish yellow solid.

To a stirred −10° C. DMF solution (45 mL) containing 3-isopropyl-4-methoxyphenol (10 g, 60 mmol) under N₂ was added 60% NaH in oil (3.36 g, 84 mmol) in portions. Once H₂ gas evolution ceased over a 30 min period as the reaction warmed to 5° C., a solution of 3,5-dibromo-4-iodonitrobenzene (24.5 g, 60 mmol) in THF (85 mL) was added over 15 min. The resulting thick slurry necessitated addition of an additional 25 mL of both DMF and THF. After stirring for 20 hr at 20° C., the reaction was quenched by cautious addition of H₂O prior to partitioning between EtOAc (500 mL) and H₂O (650 mL). The combined EtOAc fractions from two extractions were washed with sat'd NH₄Cl, dried over MgSO₄ and concentrated to dryness under vacuum to yield 27.1 g of 3-isopropyl-4-methoxy-2′,6′-dibromo-4′-nitrodiphenyl ether.

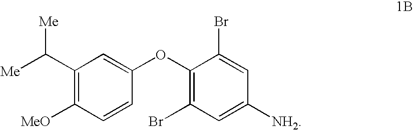

The 3-isopropyl-4-methoxy-2′,6′-dibromo-4′-nitrodiphenyl ether of Part 1A (15.2 g, 34.15 mmol) was dissolved in 129 mL of glacial HOAc and 13 mL of H₂O. Iron powder (Aldrich <10 micron, 12 g, 215 mmol) was added and the reaction was stirred under argon overnight. The reaction mixture was filtered through Celite and the pad was washed through with about 50 mL of HOAc. The filtrate was concentrated to about 60 mL and poured onto 400 g of Na₂CO₃. H₂O (400 mL) was added and the product was extracted with EtOAc (3×500 mL each). After concentration of the EtOAc layers, the residue (13.2 g) was chromatographed on Merck silica gel eluting with 25% EtOAc:hexane mixture). The desired aniline (8.75 g) was obtained in 61.7% yield as a solid.

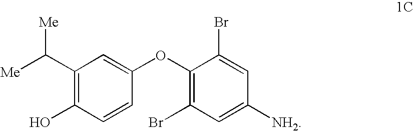

The aniline of Part 1B (8.1 g, 19.7 mmol) was dissolved in 20 mL of CH₂Cl₂ and this solution was added dropwise to a precooled (about −60° C.) solution of BBr₃ (18 mL, ca. 10 equivalents) in 180 mL of CH₂Cl₂ under argon. At this low temperature a solid precipitated. The reaction was allowed to warm up slowly to 0° C. and then stirred at 0° C. for one hour. The reaction was diluted with 200 mL of CH₂Cl₂ and quenched by pouring into a cooled, vigorously stirred solution of saturated aqueous Na₂CO₃ (300 mL) and CH₂Cl₂ (300 mL). The organic layer was separated, diluted with 100 mL of MeOH and concentrated in vacuo and taken up in MeOH (100 mL) and re-concentrated three times. The residue was dissolved in 400 mL of EtOAc, washed 2× with sat'd NaHCO₃, brine, dried (Na₂SO₄), filtered and concentrated in vacuo to yield the free phenol as a solid (7.2 g, 91% yield).

1D. Cyclobutane-1,1-dicarboxylic acid, monoacid chloride monoethyl ester

To a stirred 20° C. solution of KOH (560 mg, 10 mmol) in EtOH (20 mL) containing 180 mg H₂O was added dropwise diethyl cyclobutane-1,1-dicarboxylate (2 g, 10 mmol). After stirring for 16 hr, the reaction was concentrated under vacuum prior to quenching with 1N HCl. After addition of Et₂O, the organic layer was washed with aq. NH₄Cl, dried over Na₂SO₄ and concentrated to yield 1.6 g of the desired half ester, half acid as a light yellow oil. The latter was converted as needed to the desired half acid chloride.

To a stirred solution of half ester, half acid (124 mg, 0.72 mmol) in CH₂Cl₂ (4 mL) was added 2M oxalyl chloride/CH₂Cl₂ (0.54 mL, 1.1 mmol) followed by one drop of DMF. After stirring for 1 hr, the volatiles were removed under vacuum and the product used directly.

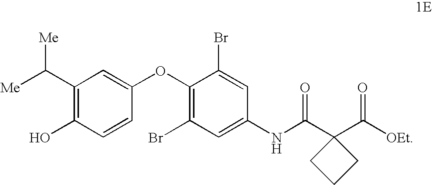

To a stirred 0° C. solution of aniline from Part 1C (250 mg, 0.62 mmol) and NaHCO₃ (167 mg, 2 mmol) in 4 mL of 3:1 of THF/H₂O, was added cyclobutane-1,1-dicarboxylic acid, monoacid chloride monoethyl ester (135 mg, 0.72 mmol), as prepared in Part 1D, in 2mL of THF. After stirring for 30 min at 0° C., the reaction was stirred for 3 hours at 20° C. THF was removed using a rotary evaporator, prior to dilution with Et₂O. The organic layer was washed with aq. NH₄CL and brine before drying over Na₂SO₄. After removal of the volatiles under vacuum, the residue was chromatographed on silica gel using 10% EtOAc/hexane to elute the desired product (260 mg) in 76% yield.

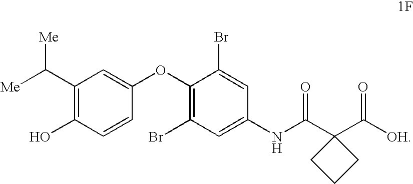

To a stirred 0° C. solution of the malonic ester from Part 1E (260 mg, 0.47 mmol) in 3 mL of 2:1 THF/H₂O was added LiOH.H₂O (42 mg, 1 mmol). The reaction reached completion upon warming slowly to 20° C. over 1.5 hr. Following removal of THF using a rotary evaporator and addition of 1.5 mL of H₂O, the reaction mixture was extracted once with 3 mL of 1:1 EtOAc/hexane. After the organic layer was washed with 2 mL of 0.05M aq. LiOH, the aqueous layers were combined. Once the pH was adjusted to 1 using 1N HCl, the suspension was extracted 3× with EtOAc. After drying over Na₂SO₄ and concentration, the desired final product was isolated as a light yellow oil. The latter was induced to foam under vacuum to yield a yellow solid (230 mg, 93%) that was used without further purification.

1H NMR (400 MHz, CDCl₃) δ 8.56 (s, 1H), 7.87 (s, 2H), 6.78 (d, J=3.1 Hz, 1H), 6.62 (d, J=8.8 Hz, 1H), 6.34 (dd, J=3.1, 8.8 Hz, 1H), 3.17 (m, 1H), 2.68-2.75 (m, 4H), 2.08 (m, 2H), 1.20 (d, J=7.0 Hz, 6H).

13C NMR (400 MHz, CDCl₃) δ 177.85, 168.84, 150.79, 148.00, 146.24, 136.25, 135.87, 124.28, 118.70, 115.70, 113.92, 112.06, 53.43, 29.61, 27.24, 22.40, 16.12.

HPLC: LUNA 4.6×50 mm, 0 to 100% B over 4 min, 4 ml/min, 1 min hold time, A=10% methanol/water+10 mm NH₄OAc, B=90% methanol/water+10 mm NH₄OAc, retention time=3.00 min.

LCMS found 526.11 (M+H)+. LRMS found 525.9 (M−H)−.

### EXAMPLE 2

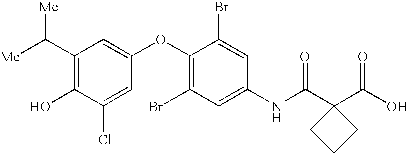

### N-[3,5-dibromo-4-[5-chloro-4-hydroxy-3-(1-methylethyl)-phenoxy]phenyl]-1-carbamyl cyclobutane-1-carboxylic acid

To a stirred solution of Example 1 (20 mg, 0.038 mmol) in 2 mL of MeCN at −30° C. was added t-butylhypochlorite (5 μL, 0.041 mmol) and aq. 1N NaHCO₃ (7.6 μL, 0.078 mmol). After stirring 1 hr at −20°−30° C., the reaction was quenched by addition of 1% aq. NaHSO₃ (0.5 mL). The MeCN was removed under vacuum using a rotary evaporator prior to dilution with EtOAc. The organic layer was washed twice with sat'd NH₄Cl, then brine prior to drying over MgSO₄. After removal of volatiles, the residue was purified by prep HPLC using MeCN/H₂O containing 0.1% TFA to elute 15 mg of desired product.

¹H NMR (400 MHz, d6-acetone) δ 9.24 (s, 1H), 8.18 (s, 2H), 6.78 (d, J=3.1 Hz, 1H), 6.58 (d, J=3.1 Hz, 1H), 3.35 (m, 1H), 2.59-2.80 (m, 4H), 2.04 (m, 2H), 1.20 (d, J=6.6 Hz, 6H).

¹³C NMR (400 MHz, d6-acetone) δ 175.06, 171.87, 152.14, 147.12, 146.45, 140.42, 125.75, 122.57, 119.82, 114.46, 114.31, 109.44, 56.18, 31.42, 30.01, 23.78, 17.90.

HPLC: LUNA 4.6×50 mm, 0 to 100 % B over 4 min, 4 ml/min, 1 min hold time, A=10% methanol/water+10 mm NH₄OAc, B=90% methanol/water+10 mm NH₄OAc, retention time=3.75 min.

LCMS found 560.21 (M+H)⁺, 558.17 (M−H)⁻. LRMS found 559.7 (M+NH₄)⁺.

### EXAMPLE 3

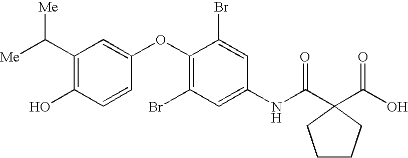

### N-[3,5-dibromo-4-[4-hydroxy-3-(1-methylethyl)-phenoxy]phenyl]-1-carbamylcyclopentane-1-carboxylic acid

In the same manner as described in Example 1, Parts E and F, the product of Example 1, Part C (250 mg, 0.62 mmol) was acylated with cyclopentane-1,1-dicarboxylic acid, monoacid chloride monoethyl ester (0.68 mmol), which had been prepared from diethyl cyclopentane-1,1-dicarboxylate, by the procedure described in Example 1, Part D. After purification by silica gel chromatography, the resulting half amide, half ester (250 mg) was subsequently converted to the desired half amide, half acid. After concentration of the EtOAc extracts, dissolution of the material in H₂O followed by lyophilization yielded 220 mg of the desired material as a fluffy white solid.

1H NMR (400 MHz, d6-acetone) δ 9.14 (s, 1H), 8.11 (s, 2H), 6.73 (d, J=4.0 Hz, 1H), 6.73 (d, J=8.0 Hz, 1H), 6.36 (dd, J=4.0, 8.0 Hz, 1H), 3.26 (m, 1H), 2.20-2.40 (m, 4H), 1.71 (m, 4H), 1.18 (d, J=7.0 Hz, 6H).

13C NMR (400 MHz, d6-acetone) δ 174.91, 171.00, 151.06, 150.21, 145.91, 138.77, 136.78, 124.64, 118.75, 116.12, 114.05, 112.74, 62.61, 35.21, 29.00, 25.99, 22.66.

HPLC: SHIMAZU VP-ODS (S-5) 4.6×50 mm, 0 to 100% B over 4 min, 4 ml/min, 1 min hold time, A=10% methanol/water+0.2% H₃PO₄, B=90% methanol/water+0.2% H₃PO₄, retention time=3.89 min.

LCMS found 540.09 (M+H)⁺.

### EXAMPLE 4

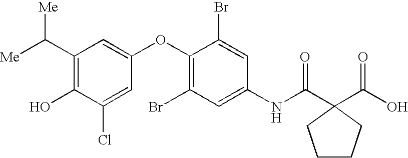

### N-[3,5-dibromo-4-[5-chloro-4-hydroxy-3-(1-methylethyl)-phenoxy]phenyl]-1-carbamylcyclopentane-1-carboxylic acid

The Example 3 compound was converted to the title compound by chlorination with t-butylhypochlorite via the procedure described in Example 2.

1H NMR (400 MHz, CD3OD) δ 7.97 (s, 2H), 6.61 (d, J=3.0 Hz, 1H), 6.45 (d, J=3.0 Hz, 1H), 3.30 (m, 1H), 2.28 (m, 4H), 1.76 (m, 4H), 1.16 (d, J=7.0 Hz, 6H).

HPLC: SHIMAZU VP-ODS (S-5) 4.6×50 mm, 0 to 100 % B over 4 min, 4 ml/min, 1 min hold time, A=10% methanol/water+0.2% H₃PO₄, B=90% methanol/water+0.2% H₃PO₄, retention time=4.07 min.

LCMS found 576.07 (M+H)⁺.

### EXAMPLE 5

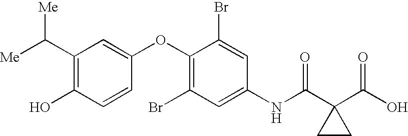

### N-[3,5-dibromo-4-[4-hydroxy-3-(1-methylethyl)-phenoxy]phenyl]-1-carbamylcyclopropane-1-carboxylic acid

In the same manner as described in Example 1, Parts E and F, the product of Example 1, Part C (250 mg, 0.62 mmol) was acylated with cyclopropane-1,1-dicarboxylic acid, monoacid chloride monoethyl ester which had been prepared from diethyl cyclopropane-1,1-dicarboxylate via the procedure described in Example 1, Part D. The resulting half amide, half ester was subsequently converted to the desired half amide, half acid.

1H NMR (400 MHz, CDCl₃) δ 10.72 (s, 1H), 7.89 (s, 2H), 6.78 (d, J=3.1 Hz, 1H), 6.62 (d, J=8.8 Hz, 1H), 6.40 (dd, J=3.1, 8.8 Hz, 1H), 3.16 (m, 1H), 2.17 (s, 2H), 1.88 (m, 4H), 1.22 (d, J=7.0 Hz, 6H).

13C NMR (400 MHz, CDCl₃) δ 177.38, 167.37, 150.82, 148.02, 146.13, 136.19, 135.91, 124.56, 118.63, 115.64, 113.89, 112.07, 27.23, 26.31, 22.39, 22.22.

HPLC: LUNA 4.6×50 mm, 0 to 100 % B over 4 min, 4 ml/min, 1 min hold time, A=10% methanol/water+10 mm NH₄OAc, B=90% methanol/water+10 mm NH₄OAc, retention time=2.94 min.

LCMS found 512.08 (M+H)⁺, 510.05 (M−H)⁻. LRMS found 511.9 (M−H)⁻.

### EXAMPLe 6

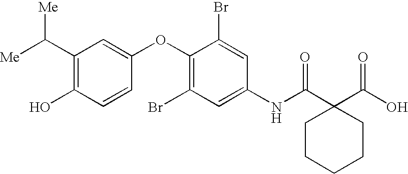

### N-[3,5-dibromo-4-[4-hydroxy-3-(1-methylethyl)-phenoxy]-phenyl]-1-carbamylcyclohexane-1-carboxylic acid

In the same manner as described in Example 1, Parts E and F, the product of Example 1, Part C (250 mg, 0.62 mmol) was acylated with cyclohexane-1,1-dicarboxylic acid, monoacid chloride monoethyl ester which had been prepared from diethyl cyclohexane-1,1-dicarboxylate via the procedure described in Example 1, Part D. The resulting half amide half ester was subsequently converted to the desired half amide, half acid.

1H NMR (400 MHz, CD3OD) δ 7.95 (s, 2H), 6.63 (d, J=3.0 Hz, 1H), 6.62 (d, J=8.0 Hz, 1H),6.32 (dd, J=3.0, 8.0 Hz, 1H), 3.23 (septet, 1H), 2.08 (m, 4H), 1.61 (m, 4H), 1.50 (m, 2H), 1.15 (d, J=7.0 Hz, 6H).

HPLC: SHIMAZU VP-ODS (S-5) 4.6×50 mm, 0 to 100 % B over 4 min, 4 ml/min, 1 min hold time, A=10% methanol/water+0.2% H₃PO₄, B=90% methanol/water+0.2% H₃PO₄, retention time=3.05 min.

LCMS found 556.0 (M+H)⁺; 554.0 (M−H)⁻.

### EXAMPLE 7

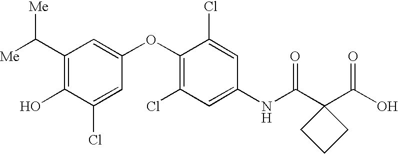

### N-[3,5-dichloro-4-[5-chloro-4-hydroxy-3-(1-methylethyl)-phenoxy]phenyl]-1-carbamylcyclobutane-1-caboxylic acid

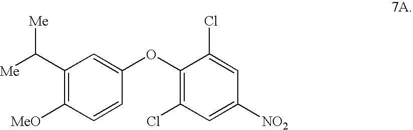

Bis-(3-isopropyl-4-methoxyphenyl)iodonium tetrafluoroborate (15.0 g, 29.4 mmol), 2,6-dichloro-4-nitrophenol (4.16 g, 20 mmol) and Cu powder [Lancaster 300 mesh (3.2 g, 50 mmol)] were suspended in 200 mL of CH₂Cl₂ in a flask covered with aluminum foil. While stirring, Et₃N (8.4 mL, 100 mmol) was added and the reaction mixture was stirred under argon in the dark for 5 days. The crude reaction mixture was concentrated to about 50 mL and then chromatographed through 2.0 liters of Merck silica gel with 3% EtOAc in hexane to yield 4.9 g (68.8%).

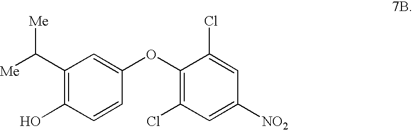

To a −78° C. solution of compound 7a (2.0 g, 5.6 mmol) in CH₂C1₂ (20 mL) was added BBr₃ (14 g, 56 mmol). After stirring for 2 hr, the reaction was quenched by addition of aq NH₄Cl followed by EtOAc. The slurry was stirred for 20 min whereupon the phases were separated. The aqueous phase was extracted 2× with EtOAc. The combined EtOAc layers were washed sequentially with NH₄Cl and brine prior to drying over MgSO₄. After removal of the solvent under vacuum, the residue was chromatographed on silica gel using 5% EtOAc/hexane to elute 1.4 g of desired phenol.

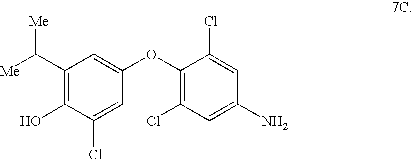

To a stirred solution of Compound 7b (600 mg, 1.7 mmol) in 4 mL of MeCN at −40° C. was added t-butylhypochlorite (230 mg, 2.1 mmol) and aq. 1N NaHCO₃ (350 μL, 0.35 mmol). After stirring 3 hr at −-20°-−30° C., the reaction was quenched by addition of 10% aq. NaHSO₃ (1 mL). The MeCN was removed under vacuum using a rotary evaporator prior to dilution with EtOAc. The organic layer was washed twice with sat'd aq. NH₄Cl followed by brine containing NH₄C1 prior to drying over Na₂SO₄. After removal of volatiles, the impure red residual oil (0.76 g) was immediately reduced by stirring with Fe powder (0.56 g, 10 mmol) in 10 mL of 1:10 H₂O/HOAc at 20° C. overnight. The reaction mixture was concentrated, diluted with EtOAc, then filtered through Celite and the pad washed thoroughly with about 50 mL of methanol. The combined filtrates were concentrated in vacuo. Saturated Na₂CO₃ (400 mL) was added and the product was extracted 3× with EtOAc. After concentration, the residue was chromatographed on silica gel using 1:4 EtOAc:hexane to elute 118 mg of desired product.

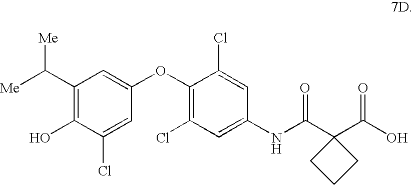

In the same manner as described in Example 1, Parts E and F, the above aniline (50 mg, 0.14 mmol) was acylated with cyclobutane-1,1-dicarboxylic acid, monoacid chloride monoethyl ester, which had been prepared from diethyl cyclobutane-1,1-dicarboxylate, by the procedure described in Example 1, Part D. After purification by silica gel chromatography using 25% EtOAc/hexane, the resulting half amide, half ester (71 mg) was subsequently hydrolyzed using aq. NaOH/EtOH to the desired half amide, half acid, which after preparative reverse phase HPLC purification employing MeCN/H₂O containing 0.1% TFA as eluent, yielded 26 mg.

1H NMR (400 MHz, CD3Od) δ 7.81 (s, 2H), 6.64 (d, J=3.1 Hz, 1H), 6.476 (d, J=3.1, 1H), 3.31 (m, 1H), 2.65 (m, 4H), 2.1 (m, 1H), 1.94 (m, 1H), 1.17 (d, J=7.0 Hz, 6H).

HPLC: LUNA 4.6×50 mm, 0 to 100% B over 4 min, 4 ml/min, 1 min hold time, A=10% methanol/water+0.2% H₃PO₄, B=90% methanol/water+0.2% H₃PO₄, retention time=4.55 min.

LCMS found 472.3, 474.35, 476.35 (M+H)⁺. LRMS found 470.3, 472.3 (M−H)⁻.

### EXAMPLE 8

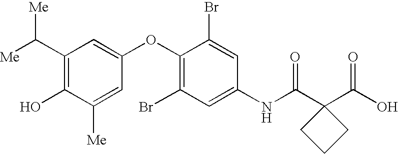

### N-[3,5-dibromo-4-[4-hydroxy-3-(1-methylethyl-5-methyl)-phenoxy]phenyl]-1-carbamylcyclobutane carboxylic acid

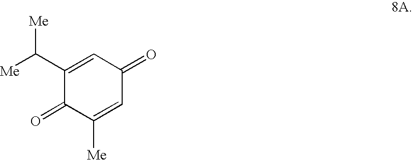

Following the procedure described N. Jacobsen, J.C.S. Perkin Trans. 1979, 2, 569, 30% aq. H₂O₂ (2.6 mL, 23.3 mmol) was added to a stirred solution of 3-isopropyl-5-methyl phenol(1 g, 6.6 mmol) in a 2.5:1 TFA/THF, at a rate to maintain 20° C. After 18 hr the brown orange solution was diluted with Et₂O and quenched by addition of solid NaHCO₃. The violet organic layer was washed repeatedly with 5% K₂CO3 until the violet color no longer remained. After drying over MgSO₄ and removal of the volatiles, 0.48 g of a yellow oil was obtained. The crude quinone was used directly since it was prone to degrade to form two more polar compounds.

8B.

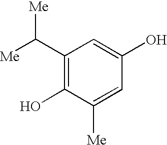

To a stirred solution of 3-isopropyl-5-methylquinone (68 mg, 0.4 mmol) in 75% aq EtOH (4 mL) was added N₂S₂O₄ (72 mg, 0.4 mmol). Heating for 1 hr at 60° C. produced approx. 50% conversion. Subsequent addition of an additional equiv. of Na₂S₂O₄ and heating for a 2^{nd} hr converted the remaining quinone to product. After dilution with aq. NH₄Cl, the reaction was extracted 3× with EtOAc. The combined EtOAc layers were washed with brine prior to drying over Na₂SO₄. The residue, after removal of the volatiles under vacuum, were chromatographed on silica gel using 15% EtOAc/hexane to elute 45 mg of desired hydroquinone as a white solid.

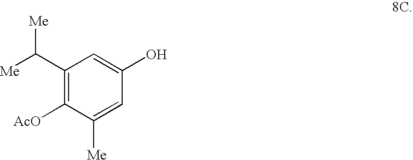

To a stirred solution of 3-isopropyl-5-methyhydroquinone (50 mg, 0.3 mmol) and DMAP (4 mg, 0.1 mmol) in pyridine (1 mL) was added AcCl (55 μL, 2.5 mmol). After slowly warming to 20° C., the reaction was stirred for 4 hr prior quenching with 1N HCl and extracting 3× with EtOAc. The residue, obtained after the combined EtOAc layers were dried over Na₂SO₄ and concentrated, was chromatographed on silica gel using 20% EtOAc/hexane to elute 69 mg of bis acetylated hydroquinone. Selective hydrolysis of the above bis acetate (50 mg, 0.3 mmol)in EtOH (1 mL) was achieved by slowly adding a solution of NaOH (12 mg, 0.29 mmol) and Na₂S₂O₄ (13 mg, 0.75 mmol) in H₂O (0.1 mL) to. After 30 min, the reaction was quenched by addition of 1N HCl followed by removal of EtOH under vacuum. The residue, after dissolution in EtOAc, was washed with NH₄Cl followed by brine prior to drying over MgSO₄. After removal of the volatiles, chromatography on silica gel with 15% EtOAc/hexane eluted 45 mg of desired 3-isopropyl-5-methyl-4-acetoxyphenol.

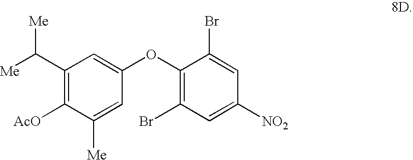

A stirred mixture of K₂CO₃ (271 mg, 1.96 mmol), 3-isopropyl-5-methyl-4-acetoxyphenol from Part 8c (371 mg, 1.78 mmol) and 4-iodo-3,5-dibromonitrobenzene (724 mg, 178 mmol, Example 1, Part A) in DMF (37 mL) was heated for 16 hr at 70° C. whereupon TLC analysis revealed the reaction to be complete. After dilution with Et₂O and 1N HCl, the mixture was extracted 2× with Et₂O. The combined Et₂O layers were washed with NH₄Cl followed by brine prior to drying over MgSO₄. After removal of the volatiles chromatography on silica gel with 15% EtOAc/hexane eluted 747 mg of desired diaryl ether as a white solid.

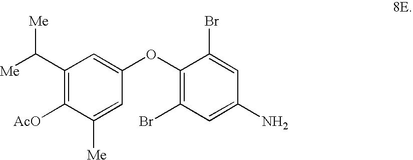

To a stirred solution of nitro diaryl ether prepared in Example 8, Part D (747 mg, 1.53 mmol) in a 1:9 H₂O/AcOH (40 mL) was added Fe powder (428 mg, 7.67 mmol). After stirring for 3 hr at 20° C., the starting material was deemed consumed by HPLC analysis. Once the ACOH was removed under vacuum, the residue was diluted with EtOAc and H₂O and extracted 2× with EtOAc. The combined EtOAc layers, after being washed with aq. NaHCO₃ and NH₄Cl and dried over MgSO₄, were concentrated to yield 726 mg of product as a white foam that was used without further purification.

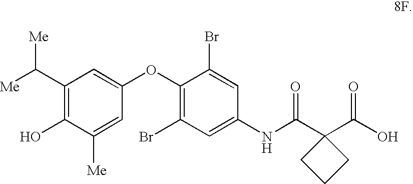

### N-[3,5-dibromo-4-[4-hydroxy-3-(1-methylethyl-5-methyl)-phenoxy]phenyl]-1-carbamylcyclo butane carboxylic acid

To a stirred 20° C. solution of 4-aminodiaryl ether of compound 8e (726 mg, 1.59 mmol) in THF (1 mL) was sequentially added 1 mL of THF containing 1-carboethoxycyclobutane-1-carbonyl chloride, previously prepared by treatment of 1-carboethoxycyclobutane-1-carboxylic acid (410 mg, 2.38 mmol) with oxalyl chloride as described in Example 1, Part D, followed by Et₃N (402 mg, 3.97 mmol). After stirring overnight, sat'd aq. NH₄Cl (5 mL) was added to quench, whereupon the volatiles were removed under vacuum. The resulting residue was dissolved in EtOAc/aq. NH₄Cl. After two EtOAc extractions, the combined EtOAc fractions were washed with aq. NH₄Cl, dried over MgSO₄ and concentrated to yield 1.08 g of a brown glass which was not further purified. The crude product was dissolved in 4:1 THF/H₂O (60 mL) and LiOH.H₂O (370 mg, 8.8 mmol) was added. After stirring the resulting solution overnight at 40° C., the volatiles were removed under vacuum. The residue was dissolved in EtOAc/1N aq. HCl. After two EtOAc extractions, the combined EtOAc fractions were washed with aq. NH₄Cl, dried over MgSO₄ and concentrated to yield 903 mg of an orange foam, which was purified by preparative HPLC using C18 reverse phase with MeCN/H₂O containing 0.1% TFA as the eluent. After recycling of the mixed fractions and concentration under vacuum, a total of 440 mg of desired product was isolated as a pale orange foam.

1H NMR (400 MHz, d6-acetone) δ 9.19 (s, 1H), 8.13 (s, 2H), 6.56 (d, J=3.1 Hz, 1H), 6.36 (d, J=3.1 Hz, 1H), 3.31 (septet, J=7 Hz, 1H), 2.69 (m, 2H), 2.57 (m, 2H), 2.18 (s, 3H), 2.04 (m, 1H), 1.94 (m, 2H), 1.14 (d, J=7.0 Hz, 6H).

HPLC: Phenominex 4.6×50 mm, 0 to 100% B over 8 min, 2.5 ml/min, 3 min hold time, A=10% methanol/water+0.2% H₃PO₄, B=90% methanol/water+0.2% H₃PO₄, retention time=8.17 min.

LRMS found 537.8, 539.7, 541.7 (M−H)⁻.

### EXAMPLE 9

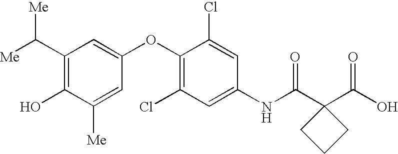

### N-[3,5-dichloro-4-[4-hydroxy-3-(1-methylethyl-5-methyl)-phenoxy]phenyl]-1-carbamylcyclobutane carboxylic acid

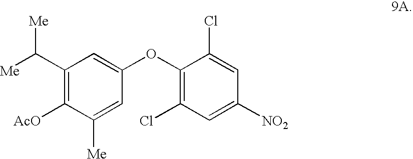

4-Acetoxy-3-isopropyl-5-methylphenol of Part 8c (50 mg, 0.24 mmol) and 3,4,5-trichloronitrobenzene (55 mg, 0.24 mmol) were heated in DMF (5 mL) at 70° C. for 16 hr to yield 85 mg of desired nitrodiaryl ether following the procedure described in Example 8, Part D.

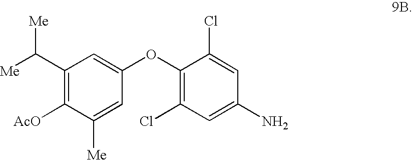

The nitrodiaryl ether prepared in Part 9A (85 mg, 0.21 mmol) was reduced with Fe powder (60 mg, 1.1 mmol) in 1:9 H₂O/AcOH (4.5 mL) following the procedure described in Example 8, Part E to yield 79 mg of product as a yellow oil that was used directly.

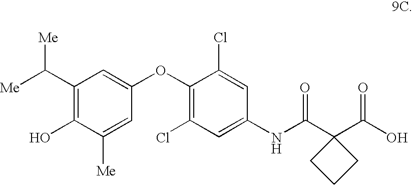

Following the procedure described in Example 1, Parts E and F, the above aniline of Part 9B (79 mg, 0.22 mmol) was acylated with cyclobutane-1,1-dicarboxylate, monoacid chloride monoethyl ester (0.25 mmol). The resulting half amide, half ester was subsequently converted to the desired half amide, half acid, which was purified by preparative HPLC employing aq. MeCN containing 0.1% TFA as a solvent to yield 50 mg of the desired final product as a white solid.

1H NMR (400 MHz, d₆-acetone) δ 9.24 (s, 1H), 7.94 (s, 2H), 6.60 (d, J=4.0 Hz, 1H), 6.38 (d, J=4.0 Hz, 1H), 3.32 (m, 1H), 2.59-2.77 (m, 4H), 2.18 (s, 3H), 1.95 (m, 2H), 1.15 (d, J=7.0 Hz, 6H).

13C NMR (400 MHz, d_{b}-acetone) δ 172.55, 169.40, 150.20, 146.83, 142.68, 136.97, 136.12, 129.02, 125.15, 119.50, 113.17, 109.86, 53.84, 28.21, 21.77, 15.76, 15.24.

HPLC: Phenominex 4.6×50 mm, 0 to 100% B over 4 min, 4 ml/min, 1 min hold time, A=10% methanol/water+10 mm NH₄OAc, B=90% methanol/water+10 mm NH₄OAc, retention time=3.53 min.

LCMS found 449.87 (M−H)⁻.

### EXAMPLE 10

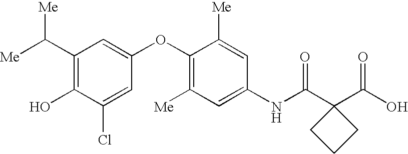

### N-[3,5-dimethyl-4-[5-chloro-4-hydroxy-3-(1-methylethyl)-phenoxy]-phenyl]-1-carbamylcyclobutanecarboxylic acid

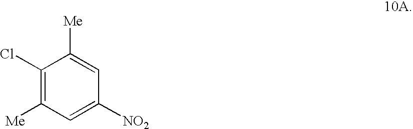

To a stirred solution of 2,6-dimethyl-4-nitrophenol (5 g, 30 mmol) in CH₂Cl₂ (50 mL) at 0° C., pyridine (2.9 mL, 36 mmol) and Tf₂O (5 mL, 36 mmol) were added sequentially. The reaction was allowed to slowly warm to 20° C. over 2 hr, whereupon after quenching with H₂O, the mixture was extracted 2× with Et₂O. The combined Et₂O layers were washed 1× with 1N NaOH, 1N HCl and brine prior to drying over MgSO₄. After removal of the volatiles using a rotary evaporator, the residue was dissolved in N-methylpyrolidone (80 mL). Following addition of LiCl (1.9 g, 45 mmol), the solution was heated at 120° C. for three days, whereupon the solvent was removed under vacuum. After addition of EtOAc and H₂O to the residue, the suspension was filtered to remove the black solids. The EtOAc layer was washed 2× with H₂O and once with brine prior to drying over MgSO₄. The dark solution was passed through a plug of silica gel before concentration. The resulting brown solid consisted of a 3:1 mixture of aryl chloride to starting phenol. Chromatography on silica gel eluting with 5% EtOAc/hexane yielded 3.3 g of 2,6-dimethyl-4-nitrochlorobenzene as white needles in 60% overall yield.

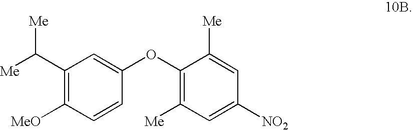

A DMF (10 mL) solution containing 2,6-dimethyl-4-nitrochlorobenzene (625 mg, 3.4 mmol)from Part 10A, K₂CO₃ (466 mg, 3.4 mmol) and 3-isopropyl-4-methoxyphenol (560 mg, 3.4 mmol) from Example 1, Part A, was heated at 120° C. for 4 days under Ar. Upon cooling, the reaction was diluted with Et₂O. The resulting solution was washed 1× with 1N HCl then aq NH₄Cl prior to drying over MgSO₄. The crude product was chromatographed on silica gel using hexane −5% EtOAc/hexane as eluent to yield 704 mg of desired diaryl ether and 126 mg of recovered 2,6-dimethyl-4-nitrochlorobenzene.

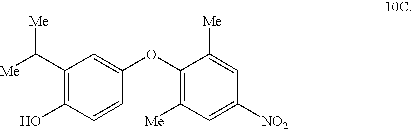

To a stirred −20° C. CH₂Cl₂ solution (6 mL) containing the diaryl ether from Part 10B (385 mg, 1.22 mmol) was added BBr₃ (0.13 mL, 1.34 mmol) dropwise. After stirring for 1 hr at less than −15° C., the reaction was maintained at 0° C. for 2 additional hours whereupon H₂O was added as a quench prior to two EtOAc extractions. The combined EtOAc layers were washed twice with aq. NH₄Cl, dried over MgSO₄ and concentrated. Chromatography of the resulting residue on silica gel eluting with 10-25% EtOAc yielded 350 mg of desired phenol.

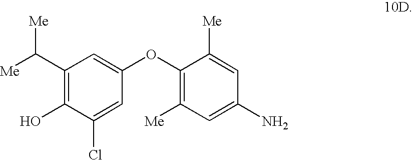

To a stirred 20° C. HOAc solution (8 mL) containing the phenolic diaryl ether of Part 10C (330 mg, 1.1 mmol) was added BnNMe₃⁺ ICL₄⁻. After 1 hr, the solids were filtered, whereupon H₂O (2 mL) and Fe dust (250 mg, 7 mmol) were added to the filtrate. The reaction was stirred for 16 hr at 20° C. and then another 2 hr at 45° C. to drive to completion. After removal of HOAc under vacuum, EtOAc and aq. NaHCO₃ were added and the suspension filtered to remove residual solids. The filtrate was washed twice with aq. NaHCO₃ prior drying over MgSO₄. Chromatography on silica gel eluting with 10-25% EtOAc yielded 291 mg of desired chlorinated aniline.

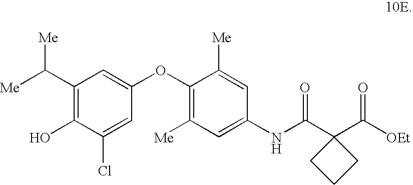

To a stirred 0° C. solution of aniline from Part 10D (290 mg, 0.95 mmol) and NaHCO₃ (200 mg, 2.5 mmol) in 9 mL of 10:1 of THF/H₂O, was added a 5 ml THF solution of cyclobutane-1,1-dicarboxylic acid, monoacid chloride monoethyl ester (188 mg, 1.1 mmol) prepared in Example 1, Part D. After stirring for 30 min, the reaction was stirred for 2 hours at 20° C. before quenching with aq. NH₄Cl. THF was removed using a rotary evaporator, prior to dilution with EtOAc. The organic layer was washed with aq. NH₄CL twice and brine before drying over MgSO₄. After removal of the volatiles under vacuum, the residue was chromatographed on silica gel using 15% EtOAc/hexane to elute the desired product (340 mg) in 78% yield.

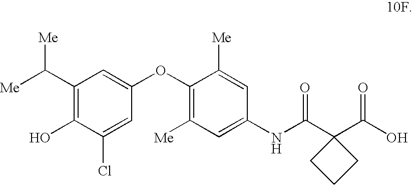

To a stirred 20° C. solution of the ethyl ester from Part 10E (325 mg, 0.71 mmol) in 6 mL of 5:1 THF/H₂O was added LiOH.H₂O (104 mg, 2.5 mmol). After 1.5 hr, the THF was removed using a rotary evaporator. The pH was adjusted to 1 using 1N HCl prior to extraction 3× with EtOAc. After washing the combined EtOAc layers 2× with aq. NH₄Cl and drying over MgSO₄, concentration yielded the desired product as a white solid (305 mg, 100%).

1H NMR (400 MHz, d6-acetone) δ 8.88 (s, 1H), 7.53 (s, 2H), 6.75 (d, J=3.1 Hz, 1H), 6.49 (d, J=3.1 Hz, 1H), 3.36 (m, 1H), 2.61-2.72 (m, 4H), 2.11 (s, 6H), 1.95 (m, 2H), 1.21 (d, J=7.0 Hz, 6H).

13C NMR (400 MHz, d6-acetone) δ 174.36, 169.92, 152.28, 147.75, 145.11, 139.34, 137.02, 132.11, 121.52, 121.08, 112.59, 112.34, 54.91, 28.61, 22.61, 16.54, 16.45.

HPLC: LUNA 4.6×50 mm, 0 to 100% B over 4 min, 4 ml/min, 1 min hold time, A=10% methanol/water+10 mm NH₄OAc, B=90% methanol/water+10 mm NH₄OAc, retention time=3.51 min.

LCMS found 432.37 (M+H)⁺. HRMS found 430.1427 (C₂₃H₂₅ClNO₅, (M−H)⁻).

It should be understood that while this invention has been described herein in terms of specific embodiments set forth in detail, such embodiments are presented by way of illustration of the general principles of the invention, and the invention is not necessarily limited thereto. Certain modifications and variations in any given material, process step or chemical formula will be readily apparent to those skilled in the art without departing from the true spirit and scope of the present invention, and all such modifications and variations should be considered within the scope of the claims that follow.

What is claimed is:

1. A compound of formula I 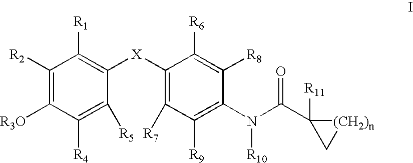
wherein
X is selected from oxygen (—O—), selenium (—Se—), sulfur (—S—), sulfenyl (SO), sulfonyl (SO₂), carbonyl (—CO), methylene (—CH₂—) and —NH—;
R₁ is selected from hydrogen, halogen, CF₃ and C₁ to C₆ alkyl;
R₂ is selected from halogen, CF₃, C₁ to C₆ alkyl, C₂ to C₆ alkenyl, C₂ to C₆ alkynyl, C₃ to C₇ cycloalkyl, C₄ to C₇ cycloalkenyl, aryl, heteroaryl, alkoxy, aryloxy, heteroaryloxy, arylalkoxy, cycloalkoxy, N(R₁₂)COR₁₃, CO(NR₁₂R₁₃), N(R₁₂)SO₂R₁₃, SO₂(NR₁₂R₁₃), SR₁₄, SOR₁₄, SO₂R₁₄, COR₁₄, CR₁₂(OR₅)R₁₃ and CH₂NR₁₂R₁₃;
R₃ is selected from hydrogen, alkyl, benzyl, aroyl and alkanoyl;
R₄ and R₅ are each independently selected from hydrogen, halogen and alkyl;
R₆ and R₇ are each independently selected hydrogen, halogen, cyano, C₁ to C₄ alkyl and C₃ to C₆ cycloalkyl, where at least one of R₆ and R₇ is other than hydrogen;
R₈ and R₉ are each independently selected from hydrogen, halogen, alkoxy, hydroxy, cyano, CF₃ and alkyl;
R₁₀ is hydrogen or alkyl;
R₁₁ is CO₂R₁₃ or tetrazole;
R₁₂ and R₁₃ for each occurrence are each independently selected from hydrogen, alkyl, cycloalkyl, aryl, heteroaryl, arylalkyl and heteroarylalkyl;
R₁₄ is selected from alkyl, cycloalkyl, aryl, heteroaryl, arylalkyl and heteroarylalkyl; and
n is an integer from 1 to 4,
including all prodrugs, stereoisomers and pharmaceutically acceptable salts thereof.

2. The compound as defined in claim 1 wherein X is oxygen.

3. The compound as defined in claim 2 wherein
R₁ is hydrogen;
R₂ is C₁ to C₆ alkyl or C₃ to C₇ cycloalkyl;
R₃ is hydrogen;
R₄ is hydrogen, halogen or alkyl;
R₅ is hydrogen;
R₆ and R₇ are each independently bromo, chloro or C₁ to C₄ alkyl;
R₈ is hydrogen, halogen or alkyl;
R₉ is hydrogen or halogen;
R₁₀ is hydrogen;
R₁₁ is carboxyl; and
n is 2 or 3.

4. The compound as defined in claim 3 wherein R₂ is isopropyl.

5. The compound as defined in claim 2 wherein
R₁ is hydrogen;
R₂ is isopropyl;
R₃ is hydrogen;
R₄ is chloro or C₁ to C₄ alkyl;
R₅ is hydrogen;
R₆ and R₇ are each independently bromo, chloro or methyl;
R₈ is hydrogen, chloro or C₁ to C₄ alkyl;
R₉ is hydrogen;
R₁₀ is hydrogen;
R₁₁ is carboxyl; and
n is 2.

6. The compound as defined in claim 2 wherein
R₁ is hydrogen;
R₂ is isopropyl;
R₃ is hydrogen;
R₄ is chloro or methyl;
R₅ is hydrogen;
R₆ and R₇ are bromo;
R₈ is hydrogen or methyl;
R₉ is hydrogen;
R₁₀ is hydrogen;
R₁₁ is carboxyl; and
n is 2.

7. The compound as defined in claim 1 having the structure 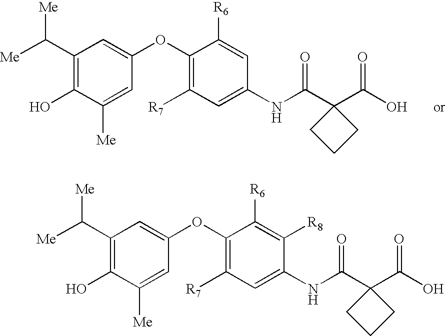
or an alkyl ester thereof.

8. The compound as defined in claim 1 having the structure 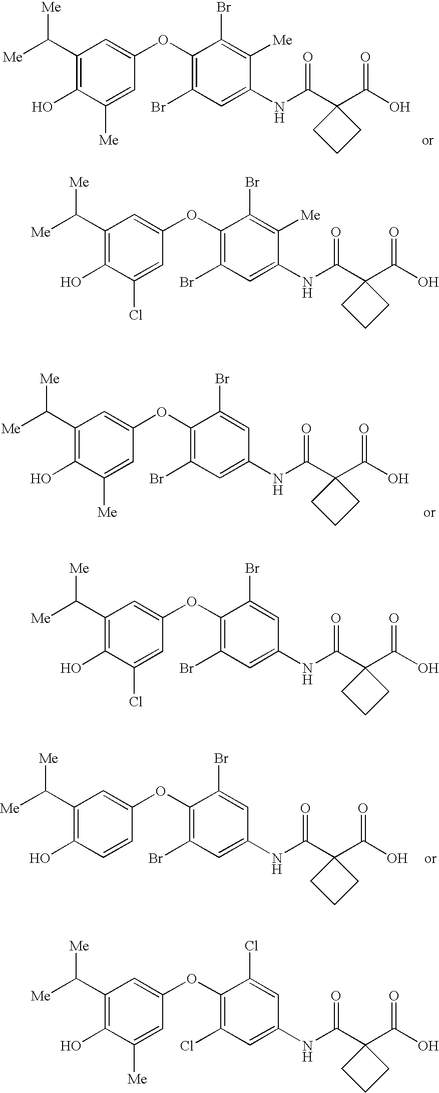
or
or an alkyl ester thereof.

9. The compound as defined in claim 1 having the structure:

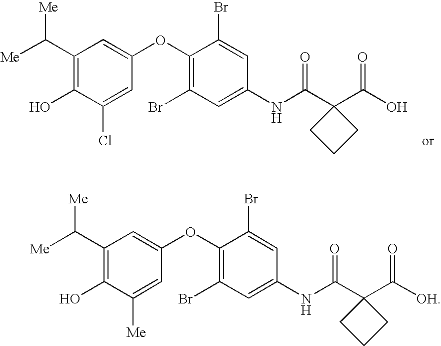

10. A pharmaceutical composition comprising a compound as defined in claim 1 and a pharmaceutically acceptable carrier therefor.

11. The pharmaceutical composition of claim 10 further comprising at least one additional therapeutic agent selected from other compounds of formula I, anti-diabetic agents, anti-osteoporosis agents, anti-obesity agents, growth promoting agents, anti-inflammatory agents, anti-anxiety agents, anti-depressants, anti-hypertensive agents, cardiac glycosides, cholesterol/lipid lowering agents, appetite supressants, bone resorption inhibitors, thyroid mimetics, anabolic agents, anti-tumor agents and retinoids.

12. The pharmaceutical composition of claim 11 wherein said additional therapeutic agent is an antidiabetic agent selected from a biguanide, a glucosidase inhibitor, a meglitinide, a sulfonylurea, a thiazolidinedione, a PPAR-alpha agonist, a PPAR-gamma agonist, a PPAR alpha/gamma dual agonist, an SGLT2 inhibitor, a glycogen phosphorylase inhibitor, an aP2 inhibitor, a glucagon-like peptide-1 (GLP-1), a dipeptidyl peptidase IV inhibitor and insulin.

13. The pharmaceutical composition of claim 11 wherein said additional therapeutic agent is an antidiabetic agent selected from metformin, glyburide, glimepiride, glipyride, glipizide, chlorpropamide, gliclazide, acarbose, miglitol, troglitazone, pioglitazone, englitazone, darglitazone, rosiglitazone and insulin.

14. The pharmaceutical composition of claim 11 wherein said additional therapeutic agent is an anti-obesity agent selected from an aP2 inhibitor, a PPAR gamma antagonist, a PPAR delta agonist, a beta 3 adrenergic agonist, a lipase inhibitor, a serotonin reuptake inhibitor, a cannabinoid-1 receptor antagonist and an anorectic agent.

15. The pharmaceutical composition of claim 11 wherein said additional therapeutic agent is a hypolipidemic agent selected from a thiazolidinedione, an MTP inhibitor, a squalene synthetase inhibitor, an HMG CoA reductase inhibitor, a fibric acid derivative, an ACAT inhibitor, a cholesterol absorption inhibitor, an ileal Na⁺/bile cotransporter inhibitor, a bile acid sequestrant and a nicotinic acid or a derivative thereof.

16. A method for preventing, inhibiting or treating a disease associated with metabolic dysfunction, or which is dependent on the expression of a T₃ regulated gene, which comprises administering to a mammalian patient in need of treatment a therapeutically effective amount of a compound as defined in claim 1.

17. A method for treating or delaying the progression or onset of obesity, hypercholesterolemia, atherosclerosis, depression, osteoporosis, hypothyroidism, subclinical hyperthyroidism, non-toxic goiter, reduced bone mass, density or growth, eating disorders, reduced cognitive function, thyroid cancer, glaucoma, cardiac arrhythmia, congestive heart failure or a skin disorder or disease, which comprises administering to mammalian patient in need of treatment a therapeutically effective amount of a compound as defined in claim 1.

18. The method according to claim 17 wherein the skin disorder or disease is dermal atrophy, post surgical bruising caused by laser resurfacing, keloids, stria, cellulite, roughened skin, actinic skin damage, lichen planus, ichtyosis, acne, psoriasis, Dernier's disease, eczema, atopic dermatitis, chloracne, pityriasis or skin scarring.

19. The method according to claim 17 further comprising administering, concurrently or sequentially, a therapeutically effective amount of at least one additional therapeutic agent selected from other compounds of formula I, anti-diabetic agents, anti-osteoporosis agents, anti-obesity agents, growth promoting agents, anti-inflammatory agents, anti-anxiety agents, anti-depressants, anti-hypertensive agents, cardiac glycosides, cholesterol/lipid lowering agents, appetite supressants, bone resorption inhibitors, thyroid mimetics, anabolic agents, anti-tumor agents and retinoids.

20. A method of treating or delaying the progression or onset of a skin disorder or disease which comprises administering to a mammalian patient a therapeutically effective amount of a compound as defined in claim 1 in combination with a retinoid or a vitamin D analog.

21. A method for treating or delaying the progression or onset of obesity which comprises administering to mammalian patient in need of treatment a therapeutically effective amount of a compound as defined in claim 1.

22. A method according to claim 21 further comprising administering, concurrently or sequentially, a therapeutically effective amount of at least one additional therapeutic agent selected from an anti-obesity agent and an appetite suppressant.

23. A method according to claim 22 wherein said anti-obesity agent is selected from aP2 inhibitors, PPAR gamma antagonists, PPAR delta agonists, beta 3 adrenergic agonists, lipase inhibitors, serotonin (and dopamine) reuptake inhibitors, cannabinoid-1 receptor antagonists, other thyroid receptor agents and anorectic agents.

24. A pharmaceutical composition which functions as a selective agonist of the thyroid hormone receptor comprising a compound as defined in claim 1.
